(19) United States
(12) Patent Application Publication
(10) Pub. No.: US 2009/0081685 A1
(43) Pub. Date: Mar. 26, 2009

(54) METHODS AND COMPOSITIONS FOR THE DETECTION OF OVARIAN DISEASE

(75) Inventors: Wayne F. Beyer, Bahama, NC (US); Thomas M. Venetta, Durham, NC (US); John W. Groelke, Raleigh, NC (US); Rainer H. Blaesius, Chapell Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: TriPath Imaging, Inc., Burlington, NC (US)

(21) Appl. No.: 12/268,850

(22) Filed: Nov. 11, 2008

Related U.S. Application Data
(62) Division of application No. 11/177,506, filed on Jul. 8, 2005.
(60) Provisional application No. 60/586,856, filed on Jul. 9, 2004.

(51) Int. Cl. G01N 33/574; C12Q 1/28; C12Q 1/02; C12Q 1/68

(52) U.S. Cl. 435/6; 435/28; 435/29; 435/7.1

(57) ABSTRACT

Methods and compositions for identifying ovarian cancer in a patient sample are provided. The methods of the invention comprise detecting overexpression of at least one biomarker in a body sample, wherein the biomarker is selectively overexpressed in ovarian cancer. In preferred embodiments, the body sample is a serum sample. The biomarkers of the invention include any genes or proteins that are selectively overexpressed in ovarian cancer, including, for example, acute phase reactants, lipoproteins, proteins involved in the regulation of the complement system, regulators of apoptosis, proteins that bind hemoglobin, heme, or iron, cytostructural proteins, enzymes that detoxify metabolic byproducts, growth factors, and hormone transporters. In some aspects of the invention, overexpression of a biomarker of interest is detected at the protein level using biomarker-specific antibodies or at the nucleic acid level using nucleic acid hybridization techniques. Kits for practicing the methods of the invention are further provided.

### CROSS REFERENCE TO RELATED APPLICATIONS

This application is a divisional of U.S. patent application Ser. No. 11/177,506, filed Jul. 8, 2005, which claims the benefit of U.S. Provisional Application Ser. No. 60/586,856, filed Jul. 9, 2004, both of which are incorporated herein by reference in their entirety.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 364703SequenceListing.txt, a creation date of Nov. 9, 2008, and a size of 228 KB. The sequence listing filed via EFS-Web is part of the specification and is hereby incorporated in its entirety by reference herein.

### FIELD OF THE INVENTION

The present invention relates to methods and compositions for the detection of ovarian cancer.

### BACKGROUND OF THE INVENTION

Ovarian cancer is responsible for significant morbidity and mortality in populations around the world. According to data from the American Cancer Society, there are an estimated 23,400 new cases of ovarian cancer per year in the United States alone. Additionally, there are 13,900 ovarian cancer-related deaths per year making it the fifth leading cancer killer among women in the United States. Since 80% to 90% of women who develop ovarian cancer will not have a family history of the disease, research efforts have focused on developing screening and diagnostic protocols to detect ovarian cancer during early stages of the disease. However, no screening test developed to date has been shown to reduce ovarian cancer mortality.

Classification of cancers determines appropriate treatment and helps determine the prognosis. Ovarian cancers are classified according to histology (i.e., “grading”) and extent of the disease (i.e., “staging”) using recognized grade and stage systems. In grade I, the tumor tissue is well differentiated. In grade II, tumor tissue is moderately well differentiated. In grade III, the tumor tissue is poorly differentiated. Grade III correlates with a less favorable prognosis than either grade I or II. Stage I is generally confined within the capsule surrounding one (stage IA) or both (stage IB) ovaries, although in some stage I (i.e. stage IC) cancers, malignant cells may be detected in ascites, in peritoneal rinse fluid, or on the surface of the ovaries. Stage II involves extension or metastasis of the tumor from one or both ovaries to other pelvic structures. In stage IIA, the tumor extends or has metastasized to the uterus, the fallopian tubes, or both. Stage IIB involves metastasis of the tumor to the pelvis. Stage IIC is stage IIA or IIB with the added requirement that malignant cells may be detected in ascites, in peritoneal rinse fluid, or on the surface of the ovaries. In stage III, the tumor comprises at least one malignant extension to the small bowel or the omentum, has formed extrapelvic peritoneal implants of microscopic (stage IIIA) or macroscopic (<2 centimeter diameter, stage IIIB; >2 centimeter diameter, stage IIIC) size, or has metastasized to a retroperitoneal or inguinal lymph node (an alternate indicator of stage IIIC). In stage IV, distant (i.e. non-peritoneal) metastases of the tumor can be detected.

The exact duration of the various stages of ovarian cancer are not known but are believed to be at least about a year each (Richart et al., 1969, Am. J. Obstet. Gynecol. 105:386). Prognosis declines with increasing stage designation. For example, 5-year survival rates for patients diagnosed with stage I, II, III, and IV ovarian cancer are 80%-95%, 57%, 25%, and 8%, respectively. Currently, greater than about 60% of ovarian cancers are diagnosed at stage III or stage 1V, where prognosis is at its worst.

The high mortality of ovarian cancer is attributable to the lack of specific symptoms among patients in the early stages of ovarian cancer, thereby making early diagnosis difficult. Patients afflicted with ovarian cancer most often present with non-specific complaints, such as abnormal vaginal bleeding, gastrointestinal symptoms, urinary tract symptoms, lower abdominal pain, and generalized abdominal distension. These patients rarely present with paraneoplastic symptoms or with symptoms which clearly indicate ovarian cancer. Due to the absence of early warning signs, less than about 40% of patients afflicted with ovarian cancer present with stage I or stage II cancer. Management of ovarian cancer would be significantly enhanced if the disease could be detected at an earlier stage when treatments are generally much more efficacious.

Ovarian cancer may be diagnosed, in part, by collecting a routine medical history from a patient and by performing physical examination, x-ray examination, and chemical and hematological studies. Hematological tests, which may be indicative of ovarian cancer, include analyses of serum levels of CA125 and DF3 proteins and plasma levels of lysophosphatidic acid (LPA). Palpation of the ovaries and ultrasound techniques, particularly including endovaginal ultrasound and color Doppler flow ultrasound techniques, can aid in detection of ovarian tumors and differentiation of ovarian cancer from benign ovarian cysts. However, a definitive diagnosis of ovarian cancer still typically requires performing an exploratory laparotomy.

Prior use of serum CA125 level as a diagnostic marker for ovarian cancer indicated that this method exhibited insufficient specificity for use as a general screening method. Use of a refined algorithm for interpreting CA125 levels in serial retrospective samples obtained from patients improved the specificity of the method without shifting detection of ovarian cancer to an earlier stage (Skakes, 1995, Cancer 76:2004). Screening for LPA to detect gynecological cancers including ovarian cancer exhibited a sensitivity of about 96% and a specificity of about 89%. However, CA125-based screening methods and LPA-based screening methods are hampered by the presence of CA125 and LPA, respectively, in the serum of patients afflicted with conditions other than ovarian cancer. For example, serum CA125 levels are known to be associated with menstruation, pregnancy, gastrointestinal and hepatic conditions (e.g., colitis and cirrhosis), pericarditis, renal disease, and various non-ovarian malignancies. Serum LPA is known, for example, to be affected by the presence of non-ovarian gynecological malignancies. A screening method having a greater specificity for ovarian cancer than the current screening methods for CA125 and LPA could provide a population-wide screening for early stage ovarian cancer.

The ineffectiveness of transvaginal sonographic testing as a reliable screening method for ovarian cancer has also been demonstrated in clinical studies. For example, in a study evaluating the efficacy of sonographic screening in 14,469 asymptomatic women, it took an average of 5200 ultrasounds for each case of invasive cancer detected (Van Nagell, et al., 2000, Gynecol. Oncol. 77:350-356). In another study, Liede et al. employed both transvaginal sonography and CA125 to screen women at high risk for ovarian cancer (2002, J. Clin. Oncol. 20:1570-1577). Liede et al. concluded that the combined screening method was not effective in reducing morbidity or mortality from ovarian cancers. Consequently, the US Preventive Services Task Force has recommended excluding routine screening for ovarian cancer from periodic examinations (Goff, et al., 2004, JAMA 22:2710).

More recently, tumor mRNA has been compared with normal tissue mRNA to identify up-regulated genes (i.e., ovarian cancer markers) in cancer tissue using cDNA micro-arrays. Prostasin, osteopontin, HE4 and a variety of other markers have been identified through this technique. A limitation of the cDNA microarray approach, however, is that transcriptional activity in the tumor does not necessarily accurately reflect the protein level or the activity of the protein in the tissue. For example, only a small percentage of genes in lung cancer tumors exhibited a statistically significant correlation between the levels of mRNA and their corresponding proteins (Chen, et al., 2002, Clin. Cancer Res. 8:2290-2305). Additionally, numerous post-translational alterations may occur in proteins that are not reflected in changes at the RNA level.

Owing to the cost and limited sensitivity and specificity of known methods for detecting ovarian cancer, population-wide screening is not presently performed. In addition, the need to perform laparotomy in order to diagnose ovarian cancer in patients who screen positive for indications of ovarian cancer limits the desirability of population-wide screening. Thus, a compelling need exists for the development of a more sensitive and specific screening and diagnostic methodology based on the expression of gene or protein ovarian cancer markers.

In summary, the survival rate and quality of patient life are improved the earlier ovarian cancer is detected. Thus, a pressing need exists for sensitive and specific methods for detecting ovarian cancer, particularly early-stage ovarian cancer.

### SUMMARY OF THE INVENTION

Compositions and methods for diagnosing ovarian cancer are provided. The methods of the invention comprise detecting overexpression of at least one biomarker in a body sample, wherein the detection of overexpression of said biomarker specifically identifies samples that are indicative of ovarian cancer. The present method distinguishes samples that are indicative of ovarian cancer from samples that are indicative of benign proliferation. Thus, the method relies on the detection of a biomarker that is selectively overexpressed in ovarian cancer states but that is not overexpressed in normal cells or cells that are not indicative of clinical disease. In particular embodiments, the methods of the invention may facilitate the diagnosis of early-stage ovarian cancer.

The biomarkers of the invention are proteins and/or genes that are selectively overexpressed in ovarian cancer. Of particular interest are biomarkers that are overexpressed in early-stage ovarian cancer. Biomarkers include, for example, acute phase reactants (e.g., protease inhibitors and inflammatory proteins), lipoproteins, proteins involved in the regulation of the complement system, regulators of apoptosis, proteins that bind hemoglobin, heme, or iron, cytostructural proteins, enzymes that detoxify metabolic byproducts, growth factors, and hormone transporters. The detection of overexpression of the biomarker genes or proteins of the invention permits the differentiation of samples that are indicative of ovarian disease from normal cells or cells that are not indicative of clinical disease (e.g., benign proliferation).

Biomarker overexpression can be assessed at the protein or nucleic acid level. In some embodiments, immunochemistry techniques are provided that utilize antibodies to detect the overexpression of biomarker proteins in patient serum samples. In this aspect of the invention, at least one antibody directed to a specific biomarker of interest is used. Overexpression can also be detected by nucleic acid-based techniques, including, for example, hybridization. Kits comprising reagents for practicing the methods of the invention are further provided.

The methods of the invention can also be used in combination with traditional gynecological and hematological diagnostic techniques such as transvaginal sonographic screening and analysis of CA125 serum levels. Thus, for example, the immunochemistry methods presented here can be combined with CA125 analysis and transvaginal sonographic testing so that all the information from the conventional methods is conserved. In this manner, the detection of biomarkers that are selectively overexpressed in ovarian cancer can reduce the high “false positive” and “false negative” rates observed with other screening methods and may facilitate mass automated screening.

### DETAILED DESCRIPTION OF THE INVENTION

The present invention provides compositions and methods for identifying or diagnosing ovarian cancer, particularly early-stage ovarian cancer. The methods comprise the detection of the overexpression of specific biomarkers that are selectively overexpressed in ovarian cancer. That is, the biomarkers of the invention are capable of distinguishing samples that are indicative of ovarian cancer from normal samples and those not characteristic of clinical disease (e.g., benign proliferation). Methods for diagnosing ovarian cancer involve detecting the overexpression of at least one biomarker that is indicative of ovarian cancer in a body sample, particularly a serum sample, from a patient. In certain aspects of the invention, the methods permit the detection of early-stage ovarian cancer. In particular embodiments, antibodies and immunochemistry techniques are used to detect expression of the biomarker of interest. Kits for practicing the methods of the invention are further provided.

“Diagnosing ovarian cancer” is intended to include, for example, diagnosing or detecting the presence of ovarian cancer, monitoring the progression of the disease, and identifying or detecting cells or samples that are indicative of ovarian cancer. The terms diagnosing, detecting, and identifying ovarian cancer are used interchangeably herein. By “ovarian cancer” is intended those conditions classified by post-exploratory laparotomy as premalignant pathology, malignant pathology, and cancer (FIGO stages I-IV). “Early-stage ovarian cancer” refers to those disease states classified as stage I or stage II carcinoma. Early detection of ovarian cancer significantly increases 5-year survival rates.

As discussed above, a significant percentage of patients misdiagnosed by traditional diagnostic methods actually have ovarian cancer. Thus, the methods of the present invention permit the accurate diagnosis of ovarian cancer in all patient populations, including these “false positive” and “false negative” cases, and facilitate the earlier detection of ovarian cancer. Detection of ovarian cancer at early stages of the disease improves patient prognosis and quality of life. The diagnosis can be made independent of CA125 and transvaginal sonographic status, although the methods of the invention can also be used in conjunction with these conventional diagnostic screening techniques.

The methods disclosed herein provide superior detection of ovarian cancer in comparison to CA125 analysis or transvaginal sonographic screening and may permit detection of early-stage ovarian cancer. In particular aspects of the invention, the sensitivity and specificity of the present methods is equal to or greater than that of CA125 or transvaginal sonographic screening. As used herein, “specificity” refers to the level at which a method of the invention can accurately identify samples that have been confirmed as nonmalignant by exploratory laparotomy (i.e., true negatives). That is, specificity is the proportion of disease negatives that are test-negative. In a clinical study, specificity is calculated by dividing the number of true negatives by the sum of true negatives and false positives. By “sensitivity” is intended the level at which a method of the invention can accurately identify samples that have been laparotomy-confirmed as positive for ovarian cancer (i.e., true positives). Thus, sensitivity is the proportion of disease positives that are test-positive. Sensitivity is calculated in a clinical study by dividing the number of true positives by the sum of true positives and false negatives. The sensitivity of the disclosed methods for the detection of ovarian cancer is at least about 70%, preferably at least about 80%, more preferably at least about 90, 91, 92, 93, 94, 95, 96, 97, 98, 99% or more. Furthermore, the specificity of the present methods is preferably at least about 70%, more preferably at least about 80%, most preferably at least about 90, 91, 92, 93, 94, 95, 96, 97, 98, 99% or more.

The biomarkers of the invention include genes and proteins. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides.

A “biomarker” is any gene or protein whose level of expression in a tissue or cell is altered compared to that of a normal or healthy cell or tissue. Biomarkers of the invention are selective for ovarian cancer. By “selectively overexpressed in ovarian cancer” is intended that the biomarker of interest is overexpressed in ovarian cancer but is not overexpressed in conditions classified as nonmalignant, benign, and other conditions that are not considered to be clinical disease. Thus, detection of the biomarkers of the invention permits the differentiation of samples indicative of ovarian cancer from normal samples and samples that are indicative of nonmalignant and benign proliferation. In this manner, the methods of the invention permit the accurate identification of ovarian cancer, even in cases mistakenly classified as normal, nonmalignant, or benign by traditional diagnostic methods (i.e., “false negatives”), such as transvaginal sonographic screening.

The biomarkers of the invention include any gene or protein that is selectively overexpressed in ovarian cancer, as defined herein above. Such biomarkers are capable of identifying genes or proteins within a patient sample that are associated with pre-malignant, malignant, or overtly cancerous ovarian disease. Although any biomarker indicative of ovarian cancer may be used in the present invention, in preferred embodiments, the biomarker is selected from the group consisting of acute phase reactants (e.g., protease inhibitors and inflammatory proteins), lipoproteins, proteins involved in the regulation of the complement system, regulators of apoptosis, proteins that bind hemoglobin, heme, or iron, cytostructural proteins, enzymes that detoxify metabolic byproducts, growth factors, and hormone transporters. Furthermore, in particular embodiments the biomarkers are selected from the group consisting of α-1-antitrypsin, AMBP, calgranulin B, carbonic anydrase, clusterin, cofilin (non-muscle isoform), ficolin 2, ficolin 3, gelsolin, haptoglobin, haptoglobin-related biomarker, hemopexin, inter-a-trypsin inhibitor, peptidyl-prolyl cis-trans isomerase A, plasma glutathione peroxidase, platelet basic protein, serotransferrin, serum amyloid A4 protein, tetranectin, transthyretin, vitronectin and zinc-α-2-glycoprotein.

Of particular interest are biomarkers that are selectively overexpressed in early-stage ovarian cancer. By “selectively overexpressed in early-stage ovarian cancer” is intended that the biomarker of interest is overexpressed in stage I or stage II ovarian cancer states but is not overexpressed in normal samples or in conditions classified as nonmalignant, benign, and other conditions that are not considered to be clinical disease. One of skill in the art will appreciate that early-stage ovarian cancer biomarkers include those genes and proteins indicative of ovarian cancer that are initially overexpressed in stage I or stage II and whose overexpression persists throughout the advanced stages of the disease, as well as biomarkers that are only overexpressed in stage I or stage II ovarian cancer. Detection of biomarkers that are selectively overexpressed in early-stage ovarian cancer may permit the earlier detection and diagnosis of ovarian cancer and, accordingly, improve patient prognosis.

Acute phase reactant proteins are biomarkers of interest and include, for example, protease inhibitors and inflammatory proteins. Alpha-1-antitrypsin is a protease inhibitor, particularly a serine protease inhibitor. Deficiency of this enzyme is associated with emphysema and liver disease. Alpha-1-antitrypsin is a potent inhibitor of elastase and also has a moderate affinity for plasmin and thrombin. The protein is encoded by a gene (PI) located on the distal long arm of chromosome 14.

AMBP, or alpha-1-micro globulin/bikunin precursor, is an acute phase reactant and is found in many physiological fluids, including plasma, urine, and cerebrospinal fluid. AMBP exists as both a free monomer and also complexed with IgA and albumin.

Inter-alpha trypsin inhibitor 4 (plasma Kallikrein-sensitive glycoprotein) also appears to be an acute phase reactant. This protein belongs to a family of Kunitz-type protease inhibitors. Unlike other members of this protein family (e.g., H1, H2 and H3), inter-alpha trypsin inhibitor 4 lacks a bikunin chain.

Calgranulin B is associated with inflammatory cytokines and is expressed in infiltrating monocytes and granulocytes. Calgranulin B is a member of the SI00 protein family. S100 genes contain 2 EF-hand calcium-binding motifs, and at least 13 family members have been identified and are located as a cluster on chromosome 1q21. Calgranulin B likely functions in the inhibition of casein kinase, and altered expression of this protein has been found in cystic fibrosis.

In particular embodiments, biomarkers of the invention comprise proteins that are involved in lipid degradation, exchange, or transport of proteins. Apolipoprotein L1 is a secreted high density lipoprotein that binds to apolipoprotein A-I. This apolipoprotein L family member may play a role in lipid exchange and transport throughout the body, as well as in reverse cholesterol transport from peripheral cells to the liver. At least three transcript variants encoding two different isoforms of this gene have been identified.

Zinc-alpha-2-glycoprotein stimulates lipid degradation in adipocytes and causes the extensive fat losses associated with some advanced cancers. The protein may also bind polyunsaturated fatty acids.

Serum amyloid A protein and serum amyloid A-4 protein are major acute phase reactants and apolipoproteins of the HDL complex. Both proteins are expressed by the liver and secreted in the plasma. Proteins that regulate the complement system or apoptotic pathways are also of interest. Complement component C3 plays a central role in the activation of the complement system. Activation of C3 is required for both classical and alternative complement activation pathways. Patients presenting with C3 deficiency display increased susceptibility to bacterial infection. Complement factor H-related protein 2 may also be involved in regulation of the complement system. Complement factor H-related protein 2 can associate with lipoproteins and may play a role in lipid metabolism.

The ficolin family of proteins activate the complement system through the lectin pathway. The ficolin family of proteins is characterized by the presence of a leader peptide (i.e., a short N-terminal segment), followed by a collagen-like region and a C-terminal fibrinogen-like domain. The collagen-like and the fibrinogen-like domains of ficolin proteins are also found in other proteins, such as, for example, complement protein C1q, tenascins, and C-type lectins known as collectins. In human serum, there are two types of ficolins. Ficolin 2, encoded by FCN2 is predominantly expressed in the liver and has been shown to have carbohydrate binding and opsonic activities. Four transcript variants of FCN2, arising by alternative splicing and encoding different isoforms of ficolin 2, have been described. The splice variant SV0 is the most predominant. FCN2 gene transcript in the liver encodes a protein of 313 amino acids and represents the longest ficolin 2 isoform. Ficolin 3 is a thermolabile beta-2-macroglycoprotein and is a member of the ficolin/opsonin p35 lectin family. The protein, which was initially identified based on its reactivity with sera from patients with systemic lupus erythematosus, has been shown to have a calcium-independent lectin activity. The protein can activate the complement pathway in association with MASPs and sMAP, thereby aiding in host defense through the activation of the lectin pathway. Alternative splicing occurs at this locus and two variants, each encoding a distinct isoform, have been identified.

The function of clusterin is not yet clear, however, it has been associated with programmed cell death (apoptosis). Clusterin is expressed in a variety of tissues and may bind to cells, membranes, and hydrophobic proteins.

Biomarker proteins that bind to heme, hemoglobin, or iron are also of interest. Haptoglobin is expressed in liver and combines with free plasma hemoglobin. Haptoglobin prevents loss of iron through the kidneys and protects the kidneys from damage by hemoglobin, while also making the hemoglobin accessible to degradative enzymes. The haptoglobin-related protein precursor is also selectively overexpressed in early-stage ovarian cancer.

Hemopexin is a heme-binding proein that transports heme to the liver for breakdown and iron recovery, after which the free hemopexin is returned to the circulation. Hemopexin is expressed by the liver and secreted in plasma.

Serotransferrin is an iron-binding glycoprotein that transports iron from the intestine, reticuloendothelial system, and liver parenchymal cells to all proliferating cells in the body. It has an approximate molecular weight of 76.5 kDa and possesses homologous C and N-terminal domains, each of which binds one ion of ferric iron. In addition to its function in iron transport, serotransferrin may also play a physiologic role as granulocyte/pollen-binding protein (GPBP) involved in the removal of certain organic matter/allergens from serum. Biomarkers proteins that comprise the cytoskeleton or are involved in maintaining, regulating, or modulating the cytostructure of the cell (i.e., cytostructural proteins) are also used in the practice of the invention. Such cytostructural proteins include, but are not limited to, actin cytoskeleton proteins, non-collagenous matrix proteins, and proteins involved in proper protein folding. Cofilin is a widely distributed intracellular actin-modulating protein that binds and depolymerizes filamentous F-actin and inhibits the polymerization of monomeric G-actin in a pH-dependent manner. Cofilin is involved in the translocation of the actin-cofilin complex from the cytoplasm to the nucleus.

Gelsolin is a calcium-regulated, actin-modulating protein that binds to the plus (or barbed) ends of actin monomers or filaments, preventing monomer exchange by blocking or capping. Gelsolin promotes the assembly of monomers into filaments (nucleation) as well as sever filaments already formed.

Tetranectin and vitronectin are noncollagenous matrix proteins. Tetranectin binds to plasminogen and to isolated kringle 4 and may be involved in the packaging of molecules destined for exocytosis. Vitronectin is found in both serum and in tissues and promotes cell adhesion and spreading, inhibits the membrane-damaging effect of the terminal cytolytic complement pathway, and binds to several serpin serine protease inhibitors. Vitronectin is a secreted protein and exists in either a single chain form or a clipped, two chain form held together by a disulfide bond.

Peptidyl-prolyl cis-trans isomerase A catalyzes the cis-trans isomerization of proline imidic peptide bonds in oligopeptides and accelerates protein folding. It is a member of the peptidyl-prolyl cis-trans isomerase (PPIase) family. Multiple pseudogenes that map to different chromosomes have been reported. Three alternatively spliced transcript variants encoding two distinct isoforms have been observed.

Enzymes that catalyze the detoxification of metabolic byproducts are also encompassed by the biomarkers of the present invention. Carbonic anhydrase I belongs to a large family of zinc metalloenzymes (i.e. the carbonic anhydrases (CAs)), that catalyze the reversible hydration of carbon dioxide. The CAs participate in a variety of biological processes, including respiration, calcification, acid-base balance, bone resorption, and the formation of aqueous humor, cerebrospinal fluid, saliva, and gastric acid. CAs show extensive diversity in tissue distribution and in their subcellular localization. CA1 is closely linked to CA2 and CA3 genes on chromosome 8, and CA1 encodes a cytosolic protein that is predominantly expressed in erythrocytes. Transcript variants of CA1 utilizing alternative polyA sites have also been described.

Plasma glutathione peroxidase catalyzes the reduction of hydrogen peroxide, organic hydroperoxide, and lipid peroxides by reduced glutathione and functions in the protection of cells against oxidative damage. Human plasma glutathione peroxidase has been shown to be a selenium-containing enzyme and expression appears to be tissue specific.

Biomarkers of interest also include growth factors and hormone-binding proteins. Platelet basic protein is a platelet-derived growth factor that belongs to the CXC chemokine family. This growth factor is a potent chemoattractant and activator of neutrophils. Platelet basic protein has been shown to stimulate various cellular processes including, for example, DNA synthesis, mitosis, glycolysis, intracellular cAMP accumulation, prostaglandin E2 secretion, and sythesis of hyaluronic acid and sulfated glycosaminoglycan. It also stimulates the formation and secretion of plasminogen activator by synovial cells. Transthyretin is a hormone binding protein, more particularly a thyroid hormone-binding protein that likely transports thyroxine from the bloodstream to the brain.

Although the above biomarkers have been discussed in detail, any biomarker that is overexpressed in ovarian cancer may be used in the practice of the invention. In particular embodiments, the biomarkers of interest are selectively overexpressed in early-stage ovarian cancer, as defined herein above.

Although the methods of the invention require the detection of at least one biomarker in a patient sample for the detection of ovarian cancer, 2, 3, 4, 5, 6, 7, 8, 9, 10 or more biomarkers may be used to practice the present invention. It is recognized that detection of more than one biomarker in a body sample may be used to identify instances of ovarian cancer. Therefore, in some embodiments, two or more biomarkers are used, more preferably, two or more complementary biomarkers. By “complementary” is intended that detection of the combination of biomarkers in a body sample result in the successful identification of ovarian cancer in a greater percentage of cases than would be identified if only one of the biomarkers was used. Thus, in some cases, a more accurate determination of ovarian cancer can be made by using at least two biomarkers. Accordingly, where at least two biomarkers are used, at least two antibodies directed to distinct biomarker proteins will be used to practice the immunochemistry methods disclosed herein. The antibodies may be contacted with the body sample simultaneously or concurrently.

In particular embodiments, the diagnostic methods of the invention comprise collecting a body sample from a patient, contacting the sample with at least one antibody specific for a biomarker of interest, and detecting antibody binding. Samples that exhibit overexpression of a biomarker of the invention, as determined by detection of antibody binding, are deemed positive for ovarian cancer. In preferred embodiments, the body sample is a serum sample. In some aspects of the invention, the sample is a plasma sample.

By “body sample” is intended any sampling of cells, tissues, or bodily fluids in which expression of a biomarker can be detected. Examples of such body samples include but are not limited to blood, lymph, urine, gynecological fluids, biopsies, and perspiration. Body samples may be obtained from a patient by a variety of techniques including, for example, by scraping or swabbing an area or by using a needle to aspirate bodily fluids. Methods for collecting various body samples are well known in the art. In preferred embodiments, the body sample comprises serum. In one embodiment, the BD Vacutainer® SST™ Tube can be used to collect patient blood for serum analysis. The tube containing the blood is inverted to ensure mixing of clot activator additive with the patient's blood, and the resulting serum is ready within 30 minutes.

Any methods available in the art for identification or detection of the biomarkers are encompassed herein. The overexpression of a biomarker of the invention can be detected on a nucleic acid level or a protein level. In order to determine overexpression, the body sample to be examined may be compared with a corresponding body sample that originates from a healthy person. That is, the “normal” level of expression is the level of expression of the biomarker in a body sample of a human subject or patient not afflicted with ovarian cancer. Such a sample can be present in standardized form. In some embodiments, determination of biomarker overexpression requires no comparison between the body sample and a corresponding body sample that originates from a healthy person. In this situation, the biomarker of interest is overexpressed to such an extent that it precludes the need for comparison to a corresponding body sample that originates from a healthy person.

Methods for detecting biomarkers of the invention comprise any methods that determine the quantity or the presence of the biomarkers either at the nucleic acid or protein level. Such methods are well known in the art and include but are not limited to western blots, northern blots, southern blots, enzyme linked immunosorbent assay (ELISA), immunoprecipitation, immunofluorescence, flow cytometry, bead-based immunochemistry, immunochemistry, molecular imprinting, nucleic acid aptamers, nucleic acid hybridization techniques, nucleic acid reverse transcription methods, and nucleic acid amplification methods. In particular embodiments, overexpression of a biomarker is detected on a protein level using, for example, antibodies that are directed against specific biomarker proteins. These antibodies can be used in various methods such as Western blot, ELISA, or immunoprecipitation techniques.

In one embodiment, antibodies specific for biomarker proteins are utilized to detect the overexpression of a biomarker protein in a body sample. The method comprises obtaining a body sample from a patient, contacting the body sample with at least one antibody directed to a biomarker that is selectively overexpressed in ovarian cancer, and detecting antibody binding to determine if the biomarker is overexpressed in the patient sample. As noted above, a more accurate diagnosis of ovarian cancer may be obtained in some cases by detecting more than one biomarker in a patient sample. Therefore, in particular embodiments, at least two antibodies directed to two distinct biomarkers are used to detect ovarian cancer. Where more than one antibody is used, these antibodies may be added to a single sample sequentially as individual antibody reagents or simultaneously as an antibody cocktail. Alternatively, each individual antibody may be added to a separate sample from the same patient, and the resulting data pooled. One of skill in the art will recognize that the immunochemistry methods described herein may be performed manually or in an automated fashion.

In a preferred immunochemistry method of the invention, a two antibody or “sandwich” ELISA is used to detect biomarker overexpression in a patient sample. Such “sandwich” or “two-site” immunoassays are known in the art. See, for example, Current Protocols in Immunology. Indirect Antibody Sandwich ELISA to Detect Soluble Antigens, John Wiley & Sons, 1991. In this aspect of the invention, two antibodies specific to two distinct antigenic sites on a single biomarker are used. By “distinct antigenic site” is intended that the antibodies are specific for different sites on the biomarker protein of interest such that binding of one antibody does not significantly interfere with binding of the other antibody to the biomarker protein. The first antibody, known as the “capture antibody,” is immobilized on or bound to a solid support. For example, a capture antibody directed to a biomarker of interest may be covalently or noncovalently attached to a microtiter plate well, a bead, a cuvette, or other reaction vessel. In a preferred embodiment, the capture antibody is bound to a microtiter plate well. Methods for attaching an antibody to a solid support are known in the art. The body sample, particularly a serum sample, is contacted with the solid support and allowed to complex with the bound capture antibody. Unbound sample is removed, and a second antibody, known as the “detection antibody,” is added to the solid matrix. The detection antibody is specific for a distinct antigenic site on the biomarker of interest and is coupled to or labeled with a substance that provides a detectable signal. Such antibody labels are well known in the art and include various enzymes, prosthetic groups, fluorescent materials, luminescent materials, bioluminescent materials, and radioactive materials. Following incubation with the detection antibody, unbound sample is removed, and biomarker expression levels are determined by quantitation of the labeled detection antibody bound to the solid support. One of skill in the art will recognize that the capture and detection antibodies can be contacted with the body sample sequentially, as described above, or simultaneously. Furthermore, the detection antibody can be incubated with the body sample first, prior to contacting the sample with the immobilized capture antibody.

Techniques for detecting antibody binding through the use of a detectable label are well known in the art. For example, antibody binding may be detected through the use of chemical reagents that generate a detectable signal that corresponds to the level of antibody binding and, accordingly, to the level of biomarker protein expression. In some embodiments, the detection antibody is coupled to an enzyme, particularly an enzyme that catalyzes the deposition of a chromogen at the antigen-antibody binding site. Enzymes of particular interest include but are not limited to horseradish peroxidase (HRP) and alkaline phosphatase (AP). Commercial antibody detection systems may also be used to practice the invention.

The above-described immunochemistry methods and formats are intended to be exemplary and are not limiting since, in general, it will be understood that any immunochemistry method or format can be used in the present invention.

The terms “antibody” and “antibodies” broadly encompass naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody.

“Antibodies” and “immunoglobulins” (Igs) are glycoproteins having the same structural characteristics. While antibodies exhibit binding specificity to an antigen, immunoglobulins include both antibodies and other antibody-like molecules that lack antigen specificity. Polypeptides of the latter kind are, for example, produced at low levels by the lymph system and at increased levels by myelomas.

The term “antibody” is used in the broadest sense and covers fully assembled antibodies, antibody fragments that can bind antigen (e.g., Fab′, F′(ab)₂, Fv, single chain antibodies, diabodies), and recombinant peptides comprising the foregoing.

The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Antibody fragments” comprise a portion of an intact antibody, preferably the antigen-binding or variable region of the intact antibody. Examples of antibody fragments include Fab, Fab′, F(ab′)2, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two antigen-combining sites and is still capable of cross-linking antigen.

“Fv” is the minimum antibody fragment that contains a complete antigen recognition and binding site. In a two-chain Fv species, this region consists of a dimer of one heavy- and one light-chain variable domain in tight, non-covalent association. In a single-chain Fv species, one heavy- and one light-chain variable domain can be covalently linked by flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy-chain C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them.

Polyclonal antibodies can be prepared by immunizing a suitable subject (e.g., chicken, rabbit, goat, mouse, or other mammal) with a biomarker protein immunogen. The antibody titer in the immunized subject can be monitored over time by standard techniques, such as with an ELISA using immobilized biomarker protein. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Alternative to preparing monoclonal antibody-secreting hybridomas, a monoclonal antibody can be identified and isolated by screening a recombinant combinatorial immunoglobulin library (e.g., an antibody phage display library) with a biomarker protein to thereby isolate immunoglobulin library members that bind the biomarker protein. Kits for generating and screening phage display libraries are commercially available (e.g., the Pharmacia Recombinant Phage Antibody System, Catalog No. 27-9400-01; and the Stratagene SurfZAP θ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Another alternative to preparing monoclonal antibodies can occur after a protein associated with early stage ovarian cancer has been identified through proteomic techniques. Following identification, a DNA database is searched for expressed sequence tag information to determine if alternate transcripts of that protein exist. Conventional nucleic acid hybridization or amplification methods can be used to verify the presence of the genetic transcript in tumor tissue. Since the protein has already been identified through proteomic techniques, the likelihood that the genetic transcript is present in a tumor tissue is high. Once the presence is verified, the gene of interest can then be cloned and expressed in an appropriate cell expression system and the resulting specific protein is purified to homogeneity. A signal sequence can be used to facilitate secretion and isolation of biomarker proteins. Signal sequences are typically characterized by a core of hydrophobic amino acids which are generally cleaved from the mature protein during secretion in one or more cleavage events. In one embodiment, a nucleic acid sequence encoding a signal sequence can be operably linked in an expression vector to a protein of interest, such as a biomarker protein or a segment thereof. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

As described herein above, detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

The antibodies used to practice the invention are selected to have high specificity for the biomarker proteins of interest. Methods for making antibodies and for selecting appropriate antibodies are known in the art. See, for example, Celis, ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. In preferred embodiments, the antibodies are selected with the end sample type (i.e., serum preparations) in mind for binding specificity.

In some aspects of the invention, antibodies directed to specific biomarkers of interest are selected and purified via a multi-step screening process. In particular embodiments, polydomas are screened to identify biomarker-specific antibodies that possess the desired traits of specificity and sensitivity. As used herein, “polydoma” refers to multiple hybridomas. The polydomas of the invention are typically provided in multi-well tissue culture plates. In the initial antibody screening step, a tumor tissue microarray comprising multiple normal, grade I (well differentiated), grade II (moderately well differentiated), grade III (poorly differentiated) samples is generated. Methods and equipment, such as the Chemicon® Advanced Tissue Arrayer, for generating arrays of multiple tissues on a single slide are known in the art. See, for example, U.S. Pat. No. 4,820,504. Undiluted supernatants from each well containing a polydoma are assayed for positive staining using standard immunohistochemistry techniques. At this initial screening step, background, non-specific binding is essentially ignored. Polydomas producing positive results are selected and used in the second phase of antibody screening.

In the second screening step, the positive polydomas are subjected to a limiting dilution process. The resulting unscreened antibodies are assayed for positive staining of grade I, II or III samples using standard immunohistochemistry techniques. At this stage, background staining is relevant, and the candidate polydomas that only stain positive for abnormal cells (i.e., cancer cells) are selected for further analysis.

To identify antibodies that can distinguish normal samples from those indicative of ovarian cancer (i.e., grade I and above), a disease panel tissue microarray is generated. This tissue microarray typically comprises multiple normal and grade I, II and III samples. Standard immunohistochemistry techniques are employed to assay the candidate polydomas for specific positive staining of samples indicative of ovarian cancer disease only (i.e., grade I samples and above). Polydomas producing positive results and minimal background staining are selected for further analysis.

Positive-staining cultures are prepared as individual clones in order to select individual candidate monoclonal antibodies. Methods for isolating individual clones are well known in the art. The supernatant from each clone comprising unpurified antibodies is assayed for specific staining of grade I, II or III samples using the tumor and disease panel tissue microarrays described herein above. Candidate antibodies showing positive staining of ovarian disease samples (i.e., grade I and above), minimal staining of other cell types (i.e., normal samples), and little background are selected for purification and further analysis. Methods for purifying antibodies through affinity adsorption chromatography are well known in the art.

In order to identify antibodies that display maximal specific staining of ovarian cancer samples and minimal background, non-specific staining in serum samples, the candidate antibodies isolated and purified in the immunohistochemistry-based screening process above are assayed using the immunochemistry techniques of the present invention, particularly the “sandwich” ELISA described herein above.

Specifically, purified antibodies of interest are used to assay a statistically significant number of serum samples from stage I, II, III and IV ovarian cancer patients. The samples are analyzed by immunochemistry methods as described herein and classified as positive, negative, or indeterminate for ovarian cancer on the basis of positive antibody staining for a particular biomarker. Sensitivity, specificity, positive predictive values, and negative predictive values for each antibody are calculated. Antibodies exhibiting maximal specific staining of ovarian cancer serum samples with minimal background (i.e., maximal signal to noise ratio) are selected for the present invention.

Identification of appropriate antibodies results in an increase in signal to noise ratio and an increase in the clinical utility of the assay. Assay format and sample type to be used are critical factors in selection of appropriate antibodies. Biomarker antibodies that produce a maximal signal to noise ratio in an immunohistochemistry format may not work as well in immunochemistry assays, such as ELISA assays. For example, secreted biomarker proteins may not be present in tissue samples at levels that accurately reflect the levels of the same protein in serum. Additionally, serum samples comprise many proteins that may interfere with antibody binding to a biomarker of interest, and the potential problems associated with these interfering proteins must be considered during antibody selection. Thus, antibody selection requires early consideration of the assay format and the end sample type to be used.

One of skill in the art will recognize that optimization of antibody titer and detection chemistry is needed to maximize the signal to noise ratio for a particular antibody. Antibody concentrations that maximize specific binding to the biomarkers of the invention and minimize non-specific binding (or “background”) will be determined. In particular embodiments, appropriate antibody titers for use in serum preparations from patients is determined by initially testing various antibody dilutions on formalin-fixed paraffin-embedded normal and ovarian cancer tissue samples. Optimal antibody concentrations and detection chemistry conditions are first determined for formalin-fixed paraffin-embedded ovarian tissue samples. The design of assays to optimize antibody titer and detection conditions is standard and well within the routine capabilities of those of ordinary skill in the art. After the optimal conditions for fixed tissue samples are determined, each antibody is then used in serum preparations under the same conditions. Some antibodies require additional optimization to reduce background staining and/or to increase specificity and sensitivity of staining in the serum samples.

Furthermore, one of skill in the art will recognize that the concentration of a particular antibody used to practice the methods of the invention will vary depending on such factors as time for binding, level of specificity of the antibody for the biomarker protein, and the type of body sample tested. Moreover, when multiple antibodies are used, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

In other embodiments, the expression of a biomarker of interest is detected at the nucleic acid level. Nucleic acid-based techniques for assessing expression are well known in the art and include, for example, determining the level of biomarker mRNA in a body sample. Many expression detection methods use isolated RNA. Any RNA isolation technique that does not select against the isolation of mRNA can be utilized for the purification of RNA from ovarian cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The term “probe” refers to any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker of the present invention. Hybridization of an mRNA with the probe indicates that the biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the biomarkers of the present invention.

An alternative method for determining the level of biomarker mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System).

Biomarker expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

In one embodiment of the invention, microarrays are used to detect biomarker expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample.

Kits for practicing the methods of the invention are further provided. By “kit” is intended any manufacture (e.g., a package or a container) comprising at least one reagent, e.g., an antibody, a nucleic acid probe, etc. for specifically detecting the expression of a biomarker of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

In a particular embodiment, the immunocytochemistry kits of the invention additionally comprise at least two reagents, e.g., antibodies, for specifically detecting the expression of at least two distinct biomarkers. Each antibody may be provided in the kit as an individual reagent or, alternatively, as an antibody cocktail comprising all of the antibodies directed to the different biomarkers of interest.

In a preferred embodiment, kits for practicing the immunochemistry methods of the invention, particularly the “sandwich” ELISA technique, are provided. Such kits are compatible with both manual and automated immunochemistry techniques. These kits comprise at least one primary capture antibody directed to a biomarker of interest, a labeled secondary detection antibody that is specific for a distinct antigenic site on the biomarker, and chemicals for the detection of the antibody binding to the biomarker. The primary capture antibody may be provided in solution for subsequent attachment to a solid support. Alternatively, the capture antibody may be provided in a kit already bound to a solid support, such as a bead or the well of a microtiter plate. Any chemicals that detect antigen-antibody binding may be used in the practice of the invention. In some embodiments, a secondary detection antibody is conjugated to an enzyme that catalyzes the calorimetric conversion of a substrate. Such enzymes and techniques for using them in the detection of antibody binding are well known in the art. In a preferred embodiment, the kit comprises a secondary detection antibody that is conjugated to HRP. Substrates, particularly chromogens, compatible with the conjugated enzyme (e.g., tetramethylbenzidine in the case of an HRP-labeled secondary detection antibody) and solutions, such as sulfuric acid, for stopping the enzymatic reaction may be further provided. In particular embodiments, chemicals for the detection of antibody binding comprise commercially available reagents and kits.

In another embodiment, the “sandwich” ELISA kits of the invention comprise antibodies for the detection of at least two different biomarkers of interest. Such kits comprise at least two primary capture antibodies and two secondary detection antibodies directed to distinct biomarkers. The capture antibodies may be provided as individual reagents or, alternatively, as a mixture of all the antibodies directed to the different biomarkers of interest.

Positive and/or negative controls may be included in the kits to validate the activity and correct usage of reagents employed in accordance with the invention. Controls may include samples, such as tissue sections, cells fixed on glass slides, etc., known to be either positive or negative for the presence of the biomarker of interest. In a particular embodiment, the positive control is a solution comprising a biomarker protein of interest. The design and use of controls is standard and well within the routine capabilities of those of ordinary skill in the art.

In other embodiments, kits for identifying ovarian cancer comprising detecting biomarker overexpression at the nucleic acid level are further provided. Such kits comprise, for example, at least one nucleic acid probe that specifically binds to a biomarker nucleic acid or fragment thereof. In particular embodiments, the kits comprise at least two nucleic acid probes that hybridize with distinct biomarker nucleic acids.

One of skill in the art will appreciate that any or all steps in the methods of the invention could be implemented by personnel or, alternatively, performed in an automated fashion. Thus, the steps of body sample preparation, sample staining, and detection of biomarker expression may be automated. In some embodiments, the methods of the invention can be used in combination with traditional ovarian cancer screening techniques. For example, the immunochemistry techniques of the present invention can be combined with the conventional CA125 serum analysis or transvaginal sonographic screening so that all of the information from conventional methods is conserved. In this manner the detection of biomarkers can reduce the high false-positive rate of CA125 screening, reduce the high false-negative rate of transvaginal sonographic screening, and may facilitate mass automated screening. Furthermore, the methods of the invention may permit the earlier detection of ovarian cancer by providing a diagnostic test that is conducive to routine, population-wide screening.

The article “a” and “an” are used herein to refer to one or more than one (i.e., to at least one) of the grammatical object of the article. By way of example, “an element” means one or more element.

The following examples are offered by way of illustration and not by way of limitation:

### EXPERIMENTAL

### Example 1

### SELDI-TOF MS Analysis of Serum Samples for the Identification of Biomarkers Indicative of Ovarian Cancer

### Materials and Methods:

The manual fractionation of serum samples was accomplished using the Ciphergen Biosystems Protocol and Serum Fractionation Kit, K100-0007, from Ciphergen Biosystems, and pooled samples consisting of frozen Normal Human Serum, NHS Pool 1, and Ovarian Cancer Serum, OCS pool 2 (see Table 1 for individual serum sample data).

To fractionate the serum, NHS pool 1 and OCS pool 2 were thawed, brought to ambient temperature, and centrifuged (14,000×RCF) for 20 min. in a cold room (4° C.). Four×20 μl aliquots of each sample were transferred to 4×V bottom wells of Nunc microtiter plate #249952. To each well was transferred 30 μl U9 buffer (9M urea, 2% CHAPS, 50 mM Tris-HCl, pH 9) followed by shaking of the plate for 20 min. at 4° C. with an IKA-MTS mixer (600 setting). After shaking, 50 μl of the treated sample was transferred from the V bottom plate wells to a separate well in a filtration plate (Nunc, Silent Screen plate w/ liprodyne membrane, #255980) with hydrated Q Ceramic HyperD F sorbent resin. The wells of the V bottom plate were then rapidly washed with 50 μl wash buffer 1 (50 mM Tris-HCl with 0.1% octyl glucopyranoside, pH 9) and transferred to corresponding wells of the same filtration plate that had received the first 50 μl treated samples. The filtration plate was mixed for 30 min. at 4° C. Fraction 1 samples (4×100 μl for each sample type) were then collected in a collection plate with the aid of a vacuum manifold. Fresh wash buffer 1 (100 μl) was added to resin in filtration plate and followed by mixing for 10 min. at RT. Each buffer 1 wash sample was then collected by vacuum into the same collection plate well that had received the first 100 μl of wash buffer 1. These fraction 1 samples represent the combined flow-through and pH 9 elutions.

Fraction 2 was collected by first adding 100 μl wash buffer 2 (50 mM HEPES with 0.1% OGP, pH 7) to resin wells, mixing for 10 min.×RT and subsequent vacuum collection into a separate collection plate from that used above. To the same resin wells, 100 μl wash buffer 2 was again added, followed by mixing and collection under vacuum into the same wells that had received the first 100 μl wash buffer 2. These fraction 2 samples contain the pH 7 elutions.

The above process for Fraction 2 was repeated with the following buffers:

Fraction 3, wash buffer 3 (100 mM Na acetate with 0.1% OGP, pH 5)
Fraction 4, wash buffer 4 (50 mM Na acetate with 0.1% OGP, pH 4)
Fraction 5, wash buffer 5 (50 mM Na citrate with 0.1% OGP, pH 3)
Fraction 6, wash buffer 6 (33.3% isopropanol/16.7% acetonitrile/0.1% TFA)

The collection plates with fractions 1-6 were stored at −80° C. overnight prior to binding analysis.

### SELDI-TOF MS Binding Analysis

The binding of fractions 1-6 for each of the 4 NHS and 4 OCS samples to CM-10, immobilized metal affinity capture (IMAC)-30 and H50 chips (arrays of 8) were evaluated in a bioprocessor. Thus, a single array of 8 for each chip type was used for each fraction (ie., 4/NHS fractions, 4/OCS fractions). The IMAC-30 chip was first activated with 100 mM CuSO₄ for 10 min. followed by 3 washes with HPLC grade water. Arrays were then washed (3×) with specific binding buffers prior to exposure to fractions (i.e., CM-10, 100 mM Na acetate, pH 4; IMAC-30, 100 mM Na phosphate, pH 7+0.5 M NaCl; H50, 10% acetonitrile (ACN)+0.1% trifluoroacetic acid (TFA)). Each chip spot received 75 μl of its respective binding buffer followed by 25 μl of a specific fraction 1-6 (1/4 dilution). The bioprocessor was placed on a shaker for 1 hr.

Arrays were washed 3× with 150 μl of their respective binding buffer with shaking for 10 min. at each wash step. Finally, arrays were rapidly washed with HPLC H₂O and air-dried. Sinapinic acid was freshly prepared in 50% ACN and 0.05% TFA and 1.5 μl spotted on each chip surface, dried and analyzed immediately in the Ciphergen SELDI instrument. Instrument settings were as follows: high mass to 200 kDa; laser intensity at 200; detector sensitivity at 9 with mass deflector at 10 kDa. Protein Standard (C100-0007) was run in auto-calibrate mode and used as reference for sample molecular weights.

### Results

### CM-10 (Weak Cation Exchanger) Protein Profiling

Fractions 4 and 6 were of most interest with respect to the proteins bound to this chip. Fraction 4, in particular, had two prominent species that appeared elevated in OCS over NHS with molecular weights (MW) of 28 kDa and 13.9 kDa (data not shown). In addition, OCS samples had less prominent peaks, which were also elevated with MW of 17.4 kDa, 15.8 kDa and 15.1 kDa (data not shown). Note that a mass of 28 k DA is in the range of the kallikrein proteins. Fraction 6 was notable in that the protein differences seen between NHS and OCS were all in the MW range of <10 kDa (data not shown). Additionally, in this profile, the sample Human Serum Albumin peaks (i.e., both singly and doubly charged species) at 66 kDa were roughly equivalent in both the NHS and OCS samples.

### IMAC-30 Protein Profiling

Fraction 6 was most notable with this chip in its differential display (up-regulated in OCS) of proteins with MW of 56.3 kDa, 28.1-28.3 kDa and 14-14.1 kDa (data not shown). MW of approximately 56, 28 and 14 kDa are in the size range of markers FLJ10546, kallikrein and HE4, respectively. Human Serum Albumin, at 66 kDa, is seen in both samples.

### H50 (Hydrophobic) Protein Profiling

All the proteins differentially displayed by this chip surface were for the most part low MW (i.e., <10 kDa) with the exception of fraction 4, which also displayed the 28 kDa and 17.5 kDa peaks (up-regulated in OCS) (data not shown). Two proteins (7.0 and 7.5 kDa) are down-regulated in OCS compared to NHS while 3 proteins (6.4, 6.6, 6.8 kDa) are up-regulated in OCS compared to NHS. One protein at 8.1 kDa appears to be at the same levels in both NHS and OCS (data not shown).

### Example 2

### Identification of Ovarian Cancer Biomarkers in Serum Samples Using Proteomic Techniques

### Materials and Methods

Normal and ovarian cancer patient serum samples were obtained from several commercial vendors (Uniglobe, Raseda, Calif.; Diagnostic Support Services, West Yarmouth, Mass.; Impath-BCP, Franklin, Mass.; ProMedDx, Norton, Mass.) and were stored at −80° C. until use. Table 2 summarizes the commercial sources of the serum samples as well as individual donor demographic information and ovarian cancer patient disease stage. Serum pools were prepared by combining equivalent volumes of the individual serum samples comprising each pool (see Table 1). Reduction of the complexity of the serum samples was achieved either by the depletion of albumin and IgG using a standard kit (ProteoPrep Blue Albumin Depletion Kit, Sigma-Aldrich Co., St. Louis, Mo.) or through fractionation using a Q HyperD F beads, an anion exchange resin (Serum Fractionation Kit K100-0007, Ciphergen Biosystems, Fremont, Calif.). Anion exchange fractions that showed differential mass fingerprinting between ovarian and normal (control) sera by SELDI-TOF MS (Ciphergen Biosystems) were further subjected to protein precipitation using four volumes of cold acetone. Samples for 2-D gel electrophoresis were prepared by reconstitution of acetone-precipitated protein pellets or by dilution of albumin/IgG-depleted sera into a standard buffer containing 8 M urea, 2% CHAPS, 50 mM dithiothreitol, 0.2% amphloytes, and bromphenol blue (BioRad Laboratories, Inc., Hercules, Calif.). In cases where the urea in the buffer was significantly diluted, solid thiourea was added to bring the combined urea/thiourea concentration back up to 8 molar.

As described in Example 1, serum fractions were analyzed by SELDI-TOF MS, prior to 2-D gel electrophoresis, using CM-10 (weak cation exchanger), IMAC-30 (metal chelater; activated with CuSO₄), and H50 (hydrophobic surface) chips. Following binding of serum fractions, chips were washed, air dried, and then coated with sinapinic acid prepared in 50% ACN and 0.05% TFA. Chips were then analyzed by SELDI-TOF. A solution containing cytochrome C, myoglobin, carbonic anhydrase, enolase, BSA, and bovine IgG was used as a standard for peak molecular weight determinations.

2-D Gel Electrophoresis: For isoelectric focusing (IEF), processed serum samples were actively loaded onto isoelectric focusing strips (immobilized pH gradient (IPG) strips, BioRad Laboratories, Inc.) for 12 hours under low voltage using the Protean IEF Cell (BioRad Laboratories). IPG strips were either 11 or 17 cm in length and had pH ranges of 3-10 or 4-7. Rehydrated, loaded IPG strips were then isoelectric focused using preset linear voltage ramp-up programs. A 500-volt holding step was utilized for IPG strips that were not manipulated immediately at the end of the actual focusing step in order to prevent diffusion of focused proteins. Focused strips were embedded in a 0.5% agarose overlay then electrophoresed in the second dimension on small precast 4-20% or 10-20% acrylamide gels (BioRad “Criterion” gels) or large, precast 10% acrylamide gels (BioRad Laboratories “Protean II” gels). Electrophoresis was carried out at room temperature under either a constant voltage of 200 V for 45 minutes (small gels) or at a constant current of 25 mA/gel for 4.5 hours (large gels). Gels were fixed and stained using a commercial silver stain kit (Silver Stain Plus, BioRad Laboratories, Inc.).

2-D Gel Image Comparison and Selection of Spots for Excision: Gels were placed on a light box and imaged using an Olympus Camedia C-4000 ZOOM digital camera. Digital images were normalized in terms of size, colorized (red for normal serum pools and blue for ovarian cancer serum pools), and printed on hp premium inkjet transparency film using an hp deskjet 6127 printer (Hewlett-Packard). Transparencies were manually overlayed on an overhead projector and visually inspected for variations in spot (protein) distribution and patterns. Corresponding spots that varied in intensity or were either present in one sample and not the other were excised as gel plugs, sent to an outside laboratory (Jan Enghild, University of Aarhus, Denmark), and processed as outlined below for identification of protein species. Primary emphasis was placed on spots that were either: 1) present in the ovarian samples and absent in the normal samples or 2) of clearly greater intensity in the ovarian samples.

Excised Spot Protein Identification by MALDI or MS/MS: Excised gel spots were digested with trypsin overnight at 37° C. Peptides were extracted and then desalted before being applied to the MALDI target and analyzed. MALDI-TOF MS or MS/MS data was acquired using a Q-T of Ultima Global instrument (Micromass/Waters Corp., Manchester, U.K.). The mass spectrometer was calibrated over the range m/z 50-3000 using polyethylene glycol mixture (1.7 mg/ml of PEG200, PEG400, PEG600, PEG1000, and PEG2000, and 0.28 mg/ml NaI in 50% (v/v) acetonitrile). Each spectrum was calibrated using glu-fibrinopeptide B (MW=1570.6774) (Sigma) as lock mass.

For peptide fingerprinting, mass spectra are acquired in the positive-ion mode over the range 800-3000 m/z. The mass list of peptides are used to search the SwissProt/TrEMBL or NCBInr protein databases on a local Mascot server using search engine Mascot software (Matrix Sciences, London, U.K.) (REF_{—}1). The searches are performed with a peptide mass tolerance of 50 ppm, carbamidomethyl modification of cystein residues, and allowed a single missed tryptic cleavage. Only significant hits as defined by Mascot probability analysis and with at least five matches of peptide masses were accepted. Usually, the peptide mass accuracy was within 10 ppm.

Tandem mass spectrometry was performed for proteins not identified by peptide fingerprinting. An abundant MS precursor ion was selected and the MS/MS data was acquired. Argon was used as a collision gas and the collision energy required for fragmentation ranged from 50 to 120 volts depending on the peptide mass. The MS/MS data was calibrated by fixing the MS precursor ion to its m/z obtained from MS. The resulting mass list of fragmented peptides was used to search the protein databases using the search engine Mascot software (Matrix Sciences, London, U.K.) (REF_{—}1). The searches were performed with a peptide mass tolerance of 2 Da, MS/MS ion mass tolerance of 0.8 Da, carbamidomethyl modification of cystein residues, and up to one missed cleavage. For all identifications, human protein databases were used.

### Results

The resultant data were divided up into five different sets. This classification was based on the identities of the serum pools that were analyzed and the methods of reduction of sample complexity that were used for each set (Table 2).

In total, a large number of proteins were identified from tryptic digests of the excised gel spots. Although numerous functional classifications are represented, the vast majority of the identified proteins are considered to be of typically medium abundance in human serum and plasma. This is consistent with what could be expected from 2-D analysis of serum in which the albumin and immunoglobulin G fractions have been depleted prior to electrophoresis.

From the list of protein spots that were positively identified, those that were considered upregulated in ovarian cancer are listed in Table 3. Individual upregulated protein spots were visualized in 2-D gel image comparisons between the normal and ovarian samples from each data set (data not shown).

### Tables

TABLE 1
Individual serum sample data

| Serum | | Patient | | | |
|---|---|---|---|---|---|
| Pool # | Vendor | ID # | Age | Sex | STAGE |
| Normal | Uniglobe | 38048 | UNK | UNK | N/A |
| Human | Uniglobe | 38051 | UNK | UNK | N/A |
| Serum | Uniglobe | 38223 | UNK | UNK | N/A |
| (NHS) | Uniglobe | 38239 | UNK | UNK | N/A |
| Pool 1 | Uniglobe | 38452 | UNK | UNK | N/A |
| | Uniglobe | 38479 | UNK | UNK | N/A |
| Normal | ProMedDx | 10305566 | 35 | F | N/A |
| Human | ProMedDx | 10331175 | 66 | F | N/A |
| Serum | ProMedDx | 10331176 | 68 | F | N/A |
| (NHS) | ProMedDx | 10367213 | 36 | F | N/A |
| Pool 2 | ProMedDx | 10367197 | 46 | F | N/A |
| | ProMedDx | 10380219 | 30 | F | N/A |
| | ProMedDx | 10380237 | 63 | F | N/A |
| Normal | ProMedDx | 10376294 | 51 | F | N/A |
| Human | ProMedDx | 10376315 | 60 | F | N/A |
| Serum | ProMedDx | 10380221 | 57 | F | N/A |
| (NHS) | ProMedDx | 10380297 | 43 | F | N/A |
| Pool 4 | ProMedDx | 10380363 | 48 | F | N/A |
| | ProMedDx | 10380378 | 34 | F | N/A |
| Ovarian | Diagnostic Support | 616030006 | 55 | F | IV |
| Cancer | Services | | | | |
| Serum | Diagnostic Support | 616030024 | 56 | F | IV |
| (OCS) | Services | | | | |
| Pool 1 | Diagnostic Support | 616030015 | 52 | F | IIIC |
| | Services | | | | |
| | Diagnostic Support | 616030016 | 53 | F | IIIA |
| | Services | | | | |
| | Diagnostic Support | 616030011 | 50 | F | IIB |
| | Services | | | | |
| | Diagnostic Support | 616030023 | 67 | F | IIB |
| | Services | | | | |
| Ovarian | Impath-BCP | 0201-192-01310 | 44 | F | IIIC |
| Cancer | Impath-BCP | 0201-192-01332 | 63 | F | IIIC |
| Serum | Impath-BCP | 0201-192-01364 | 61 | F | IIIC |
| (OCS) | Impath-BCP | 0201-192-01427 | 66 | F | III |
| Pool 2 | Impath-BCP | 0201-192-01473 | 28 | F | III |
| | Impath-BCP | 0201-192-01479 | 32 | F | III |
| | Impath-BCP | 0201-192-01484 | 34 | F | III |
| Ovarian | Diagnostic Support | 7112030117 | 61 | F | I |
| Cancer | Services | | | | |
| Serum | Diagnostic Support | 7112030119 | 43 | F | I |
| (OCS) | Services | | | | |
| Pool 4 | Diagnostic Support | 7112030138 | 47 | F | I |
| | Services | | | | |
| | Diagnostic Support | 7112030146 | 53 | F | I |
| | Services | | | | |
| | Diagnostic Support | 7112030155 | 57 | F | I |
| | Services | | | | |
| | Diagnostic Support | 7112030160 | 34 | F | I |
| | Services | | | | |

UNK—unknown
N/A—not applicable

TABLE 2
Gel Data Sets

| Gel Data | NHS | OCS | Ovarian Cancer | Serum Complexity |
|---|---|---|---|---|
| Set | Pool # | Pool # | Stage | Reduction Method |
| I | 1 | 1 | Mixed | Albumin + |
| | | | | IgG Depletion |
| II | 1 | 1 | Mixed | AEX Fractionation |
| III | 2 | 2 | III | Albumin + |
| | | | | IgG Depletion |
| IV | 2 | 2 | III | AEX Fractionation |
| V | 4 | 4 | I | Albumin + |
| | | | | IgG Depletion |

AEX—anion exchange using Q HyperD F beads

TABLE 3
Proteins Identified as Upregulated in Ovarian
Cancer by 2-D Gel Electrophoresis

| | | Sequence | Sequence |
|---|---|---|---|
| | | Identifier for | Identifier for |
| | NCBI | nucleotide | amino acid |
| Protein | Locus | sequence | sequence |
| Alpha-1-antitrypsin | P01009 | SEQ ID NO: 1 | SEQ ID NO: 27 |
| AMBP protein | P02760 | SEQ ID NO: 2 | SEQ ID NO: 28 |
| Apolipoprotein L1 | O14791 | SEQ ID NO: 3 | SEQ ID NO: 29 |
| Calgranulin B | P06702 | SEQ ID NO: 4 | SEQ ID NO: 30 |
| Carbonic anhydrase I | P00915 | SEQ ID NO: 5 | SEQ ID NO: 31 |
| Clusterin | P10909 | SEQ ID NO: 6 | SEQ ID NO: 32 |
| Cofilin, non-muscle | P23528 | SEQ ID NO: 7 | SEQ ID NO: 33 |
| isoform | | | |
| Complement C3 | P01024 | SEQ ID NO: 8 | SEQ ID NO: 34 |
| Complement factor | P36980 | SEQ ID NO: 9 | SEQ ID NO: 35 |
| H-related protein 2 | | | |
| Ficolin 2 | Q15485 | SEQ ID NO: 10 | SEQ ID NO: 36 |
| Ficolin 3 | O75636 | SEQ ID NO: 11 | SEQ ID NO: 37 |
| Gelsolin | P06396 | SEQ ID NO: 12 | SEQ ID NO: 38 |
| Haptoglobin | P00738 | SEQ ID NO: 13 | SEQ ID NO: 39 |
| Haptoglobin-related | P00739 | SEQ ID NO: 14 | SEQ ID NO: 40 |
| protein | | | |
| Hemopexin | P02790 | SEQ ID NO: 15 | SEQ ID NO: 41 |
| Inter-alpha-trypsin | Q14624 | SEQ ID NO: 16 | SEQ ID NO: 42 |
| inhibitor | | | |
| Peptidyl-prolyl cis- | P05092 | SEQ ID NO: 17 | SEQ ID NO: 43 |
| trans isomerase A | | | |
| Plasma glutathione | P22352 | SEQ ID NO: 18 | SEQ ID NO: 44 |
| peroxidase | | | |
| Platelet basic protein | P02775 | SEQ ID NO: 19 | SEQ ID NO: 45 |
| Serotransferrin | P02787 | SEQ ID NO: 20 | SEQ ID NO: 46 |
| Serum amyloid A | P02735 | SEQ ID NO: 21 | SEQ ID NO: 47 |
| protein | | | |
| Serum amyloid A-4 | P35542 | SEQ ID NO: 22 | SEQ ID NO: 48 |
| protein | | | |
| Tetranectin | P05452 | SEQ ID NO: 23 | SEQ ID NO: 49 |
| Transthyretin | P02766 | SEQ ID NO: 24 | SEQ ID NO: 50 |
| Vitronectin | P04004 | SEQ ID NO: 25 | SEQ ID NO: 51 |
| Zinc-alpha-2- | P25311 | SEQ ID NO: 26 | SEQ ID NO: 52 |
| glycoprotein | | | |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

That which is claimed:

1. A method for diagnosing ovarian cancer in a patient, the method comprising detecting expression of at least one biomarker in a body sample, wherein the at least one biomarker is selected from the group consisting of plasma glutathione peroxidase, serum amyloid A4, and vitronectin, and wherein the detection of overexpression of the at least one biomarker specifically identifies samples that are indicative of ovarian cancer.

2. The method of claim 1, wherein the method comprises detecting expression of at least two biomarkers in a body sample, wherein the detection of overexpression of the at least two biomarkers specifically identifies samples that are indicative of ovarian cancer.

3. The method of claim 1, wherein the method comprises detecting expression of at least three biomarkers in a body sample, wherein the detection of overexpression of the at least three biomarkers specifically identifies samples that are indicative of ovarian cancer.

4. The method of claim 1, wherein detecting expression of the at least one biomarker is performed at the nucleic acid level.

5. The method of claim 4, wherein detecting expression of the at least one biomarker comprises nucleic acid hybridization.

6. The method of claim 1, wherein detecting expression of the at least one biomarker is performed at the protein level.

7. The method of claim 6, wherein detecting expression of the at least one biomarker comprises using at least one antibody to detect biomarker protein expression.

8. The method of claim 1, wherein the detection of overexpression of at least one biomarker distinguishes samples that are indicative of ovarian cancer from samples that are indicative of benign proliferation.

9. The method of claim 1, wherein the method permits the detection of early-stage ovarian cancer.

10. The method of claim 1, wherein the sample is a serum sample.

11. A method for diagnosing ovarian cancer in a patient, the method comprising:
a) obtaining a body sample from the patient;
b) contacting the sample with at least one antibody, wherein the at least one antibody specifically binds to a biomarker protein that is selectively overexpressed in ovarian cancer, and wherein the biomarker protein is selected from the group consisting of plasma glutathione peroxidase, serum amyloid A4 protein, and vitronectin; and,
c) detecting binding of the at least one antibody to the biomarker protein to detect expression of the biomarker protein, wherein the detection of overexpression of the biomarker protein specifically identifies samples that are indicative of ovarian cancer, and thereby diagnosing ovarian cancer in the patient.

12. The method of claim 11, wherein said antibody is a monoclonal antibody.

13. A method for diagnosing ovarian cancer in a patient, the method comprising:
a) obtaining a body sample from the patient;
b) contacting the sample with at least two antibodies, wherein the at least two antibodies comprise a first capture antibody that is immobilized on a solid support and a second labeled detection antibody, wherein the capture antibody and the detection antibody each specifically bind to a distinct antigenic site on a biomarker protein that is selectively overexpressed in ovarian cancer, and wherein the biomarker protein is selected from the group consisting of plasma glutathione peroxidase, serum amyloid A4 protein, and vitronectin; and,
c) detecting binding of the labeled antibody to the biomarker protein to detect expression of the biomarker protein, wherein the detection of overexpression of the biomarker protein specifically identifies samples that are indicative of ovarian cancer, and thereby diagnosing ovarian cancer in the patient.

14. A kit comprising at least one antibody, wherein said antibody specifically binds to a biomarker protein that is selectively overexpressed in ovarian cancer, and wherein said biomarker is selected from the group consisting of plasma glutathione peroxidase, serum amyloid A4 protein, and vitronectin.

15. The kit of claim 14, wherein the kit comprises at least two antibodies, wherein each of said antibodies specifically binds to a biomarker protein that is selectively overexpressed in ovarian cancer.

16. The kit of claim 14, wherein the kit comprises at least three antibodies, wherein each of said antibodies specifically binds to a biomarker protein that is selectively overexpressed in ovarian cancer.

17. The kit of claim 15, wherein the kit comprises a first capture antibody that is immobilized on a solid support and a second labeled detection antibody, wherein the capture antibody and the detection antibody each specifically bind to a distinct antigenic site on a biomarker protein that is selectively overexpressed in ovarian cancer.


SEQUENCE LISTING (52 sequences)

SEQ ID NO: 1 - DNA, 1584 bp, Homo sapiens
  CDS (233)...(1489)
aagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcttgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaccaaaataccaagtctcccctcttcatgggaaaagtggtgaatcccacccaaaaataactgcctctcgctcctcaacccctcccctccatccctggccccctccctggatgacattaaagaagggttgagctggaaaaaaaaaaaaaaaaaaa

SEQ ID NO: 2 - DNA, 1413 bp, Homo sapiens
  CDS (227)...(1285)
ccggcctcttggtactgctgaccccagccaggctacagggatcgattggagctgtccttggggctgtaattggccccagctgagcagggcaaacactgaggtcaactacaagccacaggccccttccccagcctcagttcacagctgccctgttgcagggaggcggtggcccttctgttgctagaccgagcctgtgggatataccaaggcagaggagcccatagccatgaggagcctcggggccctgctcttgctgctgagcgcctgcctggcggtgagcgctggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaagtttctctatcacaaatccaaatggaacataaccatggagtcctatgtggtccacaccaactatgatgagtatgccattttcctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgagagtccggagggctgtgctaccccaagaagaggaaggatcagggggtgggcaactggtaactgaagtcaccaagaaagaagattcctgccagctgggctactcggccggtccctgcatgggaatgaccagcaggtatttctataatggtacatccatggcctgtgagactttccagtacggcggctgcatgggcaacggtaacaacttcgtcacagaaaaggagtgtctgcagacctgccgaactgtggcggcctgcaatctccccatagtccggggcccctgccgagccttcatccagctctgggcatttgatgctgtcaaggggaagtgcgtcctcttcccctacgggggctgccagggcaacgggaacaagttctactcagagaaggagtgcagagagtactgcggtgtccctggtgatggtgatgaggagctgctgcgcttctccaactgacaactggccggtctgcaagtcagaggatggccagtgtctgtcccggggtcctgtggcaggcagcgccaagcaacctgggtccaaataaaaactaaattgtaaactcctgaaaaaaaaaaaaaaaaaaa

SEQ ID NO: 3 - DNA, 2856 bp, Homo sapiens
  CDS (162)...(1358)
actttccctttcgaattcctcggtatatcttggggactggaggacctgtctggttattatacagacgcataactggaggtgggatccacacagctcagaacagctggatcttgctcagtctctgccaggggaagattccttggaggaggccctgcagcgacatggagggagctgctttgctgagagtctctgtcctctgcatctggatgagtgcacttttccttggtgtgggagtgagggcagaggaagctggagcgagggtgcaacaaaacgttccaagtgggacagatactggagatcctcaaagtaagcccctcggtgactgggctgctggcaccatggacccagagagcagtatctttattgaggatgccattaagtatttcaaggaaaaagtgagcacacagaatctgctactcctgctgactgataatgaggcctggaacggattcgtggctgctgctgaactgcccaggaatgaggcagatgagctccgtaaagctctggacaaccttgcaagacaaatgatcatgaaagacaaaaactggcacgataaaggccagcagtacagaaactggtttctgaaagagtttcctcggttgaaaagtgagcttgaggataacataagaaggctccgtgcccttgcagatggggttcagaaggtccacaaaggcaccaccatcgccaatgtggtgtctggctctctcagcatttcctctggcatcctgaccctcgtcggcatgggtctggcacccttcacagagggaggcagccttgtactcttggaacctgggatggagttgggaatcacagccgctttgaccgggattaccagcagtaccatggactacggaaagaagtggtggacacaagcccaagcccacgacctggtcatcaaaagccttgacaaattgaaggaggtgagggagtttttgggtgagaacatatccaactttctttccttagctggcaatacttaccaactcacacgaggcattgggaaggacatccgtgccctcagacgagccagagccaatcttcagtcagtaccgcatgcctcagcctcacgcccccgggtcactgagccaatctcagctgaaagcggtgaacaggtggagagggttaatgaacccagcatcctggaaatgagcagaggagtcaagctcacggatgtggcccctgtaagcttctttcttgtgctggatgtagtctacctcgtgtacgaatcaaagcacttacatgagggggcaaagtcagagacagctgaggagctgaagaaggtggctcaggagctggaggagaagctaaacattctcaacaataattataagattctgcaggcggaccaagaactgtgaccacagggcagggcagccaccaggagagatatgcctggcaggggccaggacaaaatgcaaacttttttttttttctgagacagagtcttgctctgtcgccaagttggagtgcaatggtgcgatctcagctcactgcaagctctgcctcccgtgttcaagcgattctcctgccttggcctcccaagtagctgggactacaggcgcctaccaccatgcccagctaatttttgtatttttaatagagatggggtttcaccatgttggccaggatggtctcgatctcctgacctcttgatctgcccaccttggcctcccaaagtgctgggattacaggcgtgagccatcgcttttgacccaaatgcaaacattttattagggggataaagagggtgaggtaaagtttatggaactgagtgttagggactttggcatttccatagctgagcacagcaggggaggggttaatgcagatggcagtgcagcaaggagaaggcaggaacattggagcctgcaataagggaaaaatgggaactggagagtgtggggaatgggaagaagcagtttactttagactaaagaatatattggggggccgggtgtagtggctcatgcctgtaatccgagcactttgggaggccaaggcgggcggatcacgaggtcaggagatcgagaccatcctggctaacacagtgaaaccccgtctctactaaaaatacaaaaaattagccgggcatggtggcgggcgcctgtagttccagctaactgggcggctgaggcaggagaatggcgtgaacctgggaggtggagcttgcagtgagccgagatatcgccactgcactccagcctgggtgacagagcgagactccatctcaaaaaaaaaaaaaaaaagaatatattgacggaagaatagagaggaggcttgaaggaaccagcaatgagaaggccaggaaaagaaagagctgaaaatggagaaagcccaagagttagaacagttggatacaggagaagaaacagcggctccactacagacccagccccaggttcaatgtcctccgaagaatgaagtctttccctggtgatggtcccctgccctgtctttccagcatccactctcccttgtcctcctgggggcatatctcagtcaggcagcggcttcctgatgatggtcattggggtggttgtcatgtgatgggtcccctccaggttactaaagggtgcatgtcccctgcttgaacactgaagggcaggtggtgggccatggccatggtccccagctgaggagcaggtgtccctgagaacccaaacttcccagagagtatgtgagaaccaaccaatgaaaacagtcccatcgctcttacccggtaagtaaacagtcagaaaattagcatgaaagcagtttagcattgggaggaagctcagatctctagagctgtcttgtcgccgcccaggattgacctgtgtgtaagtcccaataaactcacctactcatcaagctgga

SEQ ID NO: 4 - DNA, 576 bp, Homo sapiens
  CDS (46)...(390)
aaacactctgtgtggctcctcggctttgggacagagtgcaagacgatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccatggccacagtcatggtggccacggccacaggccactaatcaggaggccaggccaccctgcctctacccaaccagggccccggggcctgttatgtcaaactgtcttggctgtggggctaggggctggggccaaataaagtctcttcctccaa

SEQ ID NO: 5 - DNA, 1264 bp, Homo sapiens
  CDS (147)...(932)
gtggtacccagtcctcaggtgcaaccccctgcgtggtcctctgtggcagccttctctcattcagagctgttttccacagaggtagtgaaaagaactggattttcaagttcactttgcaagagaaaaagaaaactcagtagaagataatggcaagtccagactggggatatgatgacaaaaatggtcctgaacaatggagcaagctgtatcccattgccaatggaaataaccaatcccctgttgatattaaaaccagtgaaaccaaacatgacacctctctgaaacctattagtgtctcctacaacccagccacagccaaagaaattatcaatgtggggcattctttccatgtaaattttgaggacaacgataaccgatcagtgctgaaaggtggtcctttctctgacagctacaggctctttcagtttcattttcactggggcagtacaaatgagcatggttcagaacatacagtggatggagtcaaatattctgccgagcttcacgtagctcactggaattctgcaaagtactccagccttgctgaagctgcctcaaaggctgatggtttggcagttattggtgttttgatgaaggttggtgaggccaacccaaagctgcagaaagtacttgatgccctccaagcaattaaaaccaagggcaaacgagccccattcacaaattttgacccctctactctccttccttcatccctggatttctggacctaccctggctctctgactcatcctcctctttatgagagtgtaacttggatcatctgtaaggagagcatcagtgtcagctcagagcagctggcacaattccgcagccttctatcaaatgttgaaggtgataacgctgtccccatgcagcacaacaaccgcccaacccaacctctgaagggcagaacagtgagagcttcattttgatgattctgagaagaaacttgtccttcctcaagaacacagccctgcttctgacataatccagttaaaataataatttttaagaaataaatttatttcaatattagcaagacagcatgccttcaaatcaatctgtaaaactaagaaacttaaattttagttcttactgcttaattcaaataataattagtaagctagcaaatagtaatctgtaagcataagcttatcttaaattcaagtttagtttgaggaattctttaaaattacaactaagtgatttgtatgtctatttttttcagtttatttgaaccaataaaataattttatctctttct

SEQ ID NO: 6 - DNA, 1676 bp, Homo sapiens
  CDS (48)...(1397)
gaattccgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccacagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcaccttacgggggcatcttgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgaaaaaaaaaaaaaaaaaaaaaaaaaaggaattc

SEQ ID NO: 7 - DNA, 1059 bp, Homo sapiens
  CDS (52)...(552)
gctctcgtcttctgcggctctcggtgccctctccttttcgtttccggaaacatggcctccggtgtggctgtctctgatggtgtcatcaaggtgttcaacgacatgaaggtgcgtaagtcttcaacgccagaggaggtgaagaagcgcaagaaggcggtgctcttctgcctgagtgaggacaagaagaacatcatcctggaggagggcaaggagatcctggtgggcgatgtgggccagactgtcgacgatccctacgccacctttgtcaagatgctgccagataaggactgccgctatgccctctatgatgcaacctatgagaccaaggagagcaagaaggaggatctggtgtttatcttctgggcccccgagtctgcgccccttaagagcaaaatgatttatgccagctccaaggacgccatcaagaagaagctgacagggatcaagcatgaattgcaagcaaactgctacgaggaggtcaaggaccgctgcaccctggcagagaagctggggggcagtgcggtcatctccctggagggcaagcctttgtgagccccttctggccccctgcctggagcatctggcagccccacacctgcccttgggggttgcaggctgcccccttcctgccagaccggaggggctggggggatcccagcagggggaggcaatcccttcaccccagttgccaaacagaccccccaccccctggattttccttctccctccatcccttgacggttctggccttcccaaactgcttttgatcttttgattcctcttgggctgaagcagaccaagttccccccaggcaccccagttgtgggggagcctgtattttttttaacaacatccccattccccacctggtcctcccccttcccatgctgccaacttctaaccgcaatagtgactctgtgcttgtctgtttagttctgtgtataaatggaatgttgtggagatgacccctccctgtgccggctggttcctctcccttttcccctggtcacggctactcatggaagcaggaccagtaagggaccttcgattaaaaaaaaa

SEQ ID NO: 8 - DNA, 5067 bp, Homo sapiens
  CDS (61)...(5052)
ctcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacctccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtcagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgccgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtctttgggtgccccaactgaccacacccccattcc

SEQ ID NO: 9 - DNA, 1040 bp, Homo sapiens
  CDS (78)...(890)
ggaattcggcacgagattcaaagcaacaccaccaccactgaagtatttttagttatataagattggaactaccaagcatgtggctcctggtcagtgtaattctaatctcacggatatcctctgttgggggagaagcaatgttctgtgattttccaaaaataaaccatggaattctatatgatgaagaaaaatataagccattttcccaagttcctacaggggaagttttctattactcctgtgaatataattttgtgtctccttcaaaatccttttggactcgcataacgtgcgcagaagaaggatggtcaccaacaccaaagtgtctcagactgtgtttctttccttttgtggaaaatggtcattctgaatcttcaggacaaacacatctggaaggtgatactgtacaaattatttgcaacacaggatacagacttcaaaacaatgagaacaacatttcatgtgtagaacggggctggtccactcctcccaaatgcaggtccactatttctgcagaaaaatgtgggccccctccacctattgacaatggagacattacttcattcctgttgtcagtatatgctccaggttcatcagttgagtaccagtgccagaacttgtatcaacttgagggtaacaatcaaataacatgtagaaacggacaatggtcagaaccaccaaaatgcttagatccatgtgtaatatcacaagaaattatggaaaaatataacataaaattaaagtggacaaaccaacaaaagctttattcaagaacaggtgacatagttgaatttgtttgtaaatctggatatcatccaacaaaatctcattcatttcgagcaatgtgtcagaatgggaaactggtatatcccagttgtgaggaaaaatagaatcaatggcattactattagtaaaatgcacacctttttctgaatttactattatatttgttttcaatttcatttttcaagtactgttttactcatttttattcataaataaagttttgtgttgatttgtgaaaatgcaattacaaaaaa

SEQ ID NO: 10 - DNA, 1058 bp, Homo sapiens
  CDS (11)...(952)
accagaagagatggagctggacagagctgtgggggtcctgggcgctgccaccctgctgctctctttcctgggcatggcctgggctctccaggcggcagacacctgtccagaggtgaagatggtgggcctggagggctctgacaagctcaccattctccgaggctgtccggggctgcctggggcccctggcgacaagggagaggcaggcaccaatggaaagagaggagaacgtggcccccctggacctcctgggaaggcaggaccacctgggcccaacggagcacctggggagccccagccgtgcctgacaggcccgcgtacctgcaaggacctgctagaccgagggcacttcctgagcggctggcacaccatctacctgcccgactgccggcccctgactgtgctctgtgacatggacacggacggagggggctggaccgttttccagcggagggtggatggctctgtggacttctaccgggactgggccacgtacaagcagggcttcggcagtcggctgggggagttctggctggggaatgacaacatccacgccctgaccgcccagggaaccagcgagctccgtgtagacctggtggactttgaggacaactaccagtttgctaagtacagatcattcaaggtggccgacgaggcggagaagtacaatctggtcctgggggccttcgtggagggcagtgcgggagattccctgacgttccacaacaaccagtccttctccaccaaagaccaggacaatgatcttaacaccggaaattgtgctgtgatgtttcagggagcttggtggtacaaaaactgccatgtgtcaaacctgaatggtcgctacctcagggggactcatggcagctttgcaaatggcatcaactggaagtcggggaaaggatacaattatagctacaaggtgtcagagatgaaggtgcgacctgcctagcccaggccggcctcagggtcaggacgcctccacacatagttggttggggggtagggtttgggagcttggccctacggtttgtaaaagaaacacatgtcgtgattct

SEQ ID NO: 11 - DNA, 1059 bp, Homo sapiens
  CDS (7)...(906)
agcaagatggatctactgtggatcctgccctccctgtggcttctcctgcttggggggcctgcctgcctgaagacccaggaacaccccagctgcccaggacccagggaactggaagccagcaaagttgtcctcctgcccagttgtcccggagctccaggaagtcctggggagaagggagccccaggtcctcaagggccacctggaccaccaggcaagatgggccccaagggtgagccaggagatccagtgaacctgctccggtgccaggaaggccccagaaactgccgggagctgttgagccagggcgccaccttgagcggctggtaccatctgtgcctacctgagggcagggccctcccagtcttttgtgacatggacaccgaggggggcggctggctggtgtttcagaggcgccaggatggttctgtggatttcttccgctcttggtcctcctacagagcaggttttgggaaccaagagtctgaattctggctgggaaatgagaatttgcaccagcttactctccagggtaactgggagctgcgggtagagctggaagactttaatggtaaccgtactttcgcccactatgcgaccttccgcctcctcggtgaggtagaccactaccagctggcactgggcaagttctcagagggcactgcaggggattccctgagcctccacagtgggaggccctttaccacctatgacgctgaccacgattcaagcaacagcaactgtgcagtgattgtccacggtgcctggtggtatgcatcctgttaccgatcaaatctcaatggtcgctatgcagtgtctgaggctgccgcccacaaatatggcattgactgggcctcaggccgtggtgtgggccacccctaccgcagggttcggatgatgcttcgatagggcactctggcagccagtgcccttatctctcctgtacagcttccggatcgtcagccaccttgcctttgccaaccacctctgcttgcctgtccacatttaaaaataaaatcattttagccctttcaaaaaaaaaaaaaaaaaaaaaaaaaaaaa

SEQ ID NO: 12 - DNA, 2705 bp, Homo sapiens
  CDS (48)...(2396)
acccgaggccgcggctgccgactgggtcccctgccgctgtcgccaccatggctccgcaccgccccgcgcccgcgctgctttgcgcgctgtccctggcgctgtgcgcgctgtcgctgcccgtccgcgcggccactgcgtcgcggggggcgtcccaggcgggggcgccccaggggcgggtgcccgaggcgcggcccaacagcatggtggtggaacaccccgagttcctcaaggcagggaaggagcctggcctgcagatctggcgtgtggagaagttcgatctggtgcccgtgcccaccaacctttatggagacttcttcacgggcgacgcctacgtcatcctgaagacagtgcagctgaggaacggaaatctgcagtatgacctccactactggctgggcaatgagtgcagccaggatgagagcggggcggccgccatctttaccgtgcagctggatgactacctgaacggccgggccgtgcagcaccgtgaggtccagggcttcgagtcggccaccttcctaggctacttcaagtctggcctgaagtacaagaaaggaggtgtggcatcaggattcaagcacgtggtacccaacgaggtggtggtgcagagactcttccaggtcaaagggcggcgtgtggtccgtgccaccgaggtacctgtgtcctgggagagcttcaacaatggcgactgcttcatcctggacctgggcaacaacatccaccagtggtgtggttccaacagcaatcggtatgaaagactgaaggccacacaggtgtccaagggcatccgggacaacgagcggagtggccgggcccgagtgcacgtgtctgaggagggcactgagcccgaggcgatgctccaggtgctgggccccaagccggctctgcctgcaggtaccgaggacaccgccaaggaggatgcggccaaccgcaagctggccaagctctacaaggtctccaatggtgcagggaccatgtccgtctccctcgtggctgatgagaaccccttcgcccagggggccctgaagtcagaggactgcttcatcctggaccacggcaaagatgggaaaatctttgtctggaaaggcaagcaggcaaacacggaggagaggaaggctgccctcaaaacagcctctgacttcatcaccaagatggactaccccaagcagactcaggtctcggtccttcctgagggcggtgagaccccactgttcaagcagttcttcaagaactggcgggacccagaccagacagatggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctgaggaggggcagggcccacccatgtcaccggtcagtgccttttggaactgtccttccctcaaagaggccttagagcgagcagagcagctctgctatgagtgtgtgtgtgtgtgtgtgttgtttcttttttttttttttacagtatccaaaaatagccctgcaaaaattcagagtccttgcaaaattgtctaaaatgtcagtgtttgggaaattaaatccaataaaaacattttgaagtgtgaaaaaaaaaaaaaaaaaaaaaaaaaaaaaaaaaaaaaaaaaaaaaaaaaaaaaaaaaaaaaaaaaaaaaa

SEQ ID NO: 13 - DNA, 1412 bp, Homo sapiens
  CDS (27)...(1247)
ctcttccagaggcaagaccaaccaagatgagtgccttgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccccactttaacactctatgtggggaaaaagcagcttgtagagattgagaaggttgttctacaccctaactactcccaagtagatattgggctcatcaaactcaaacagaaggtgtctgttaatgagagagtgatgcccatctgcctaccatccaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgaccca

SEQ ID NO: 14 - DNA, 1245 bp, Homo sapiens
  CDS (31)...(1077)
actgctcttccagaggcaagaccaaccaagatgagtgacctgggagctgtcatttccctcctgctctggggacgacagctttttgcactgtactcaggcaatgatgtcacggatatttcagatgaccgcttcccgaagccccctgagattgcaaatggctatgtggagcacttgtttcgctaccagtgtaagaactactacagactgcgcacagaaggagatggagtatacaccttaaatgataagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggggccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggtggttctacaccctaactaccaccaggtagatattgggctcatcaaactcaaacagaaggtgcttgttaatgagagagtgatgcccatctgcctaccttcaaagaattatgcagaagtagggcgtgtgggttacgtgtctggctggggacaaagtgacaactttaaacttactgaccatctgaagtatgtcatgctgcctgtggctgaccaatacgattgcataacgcattatgaaggcagcacatgccccaaatggaaggcaccgaagagccctgtaggggtgcagcccatactgaacgaacacaccttctgtgtcggcatgtctaagtaccaggaagacacctgctatggcgatgcgggcagtgcctttgccgttcacgacctggaggaggacacctggtacgcggctgggatcctaagctttgataagagctgtgctgtggctgagtatggtgtgtatgtgaaggtgacttccatccagcactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccaaaa

SEQ ID NO: 15 - DNA, 1389 bp, Homo sapiens
  CDS (1)...(1389)
atggctagggtactgggagcacccgttgcactggggttgtggagcctatgctggtctctggccattgccacccctcttcctccgactagtgcccatgggaatgttgctgaaggcgagaccaagccagacccagacgtgactgaacgctgctcagatggctggagctttgatgctaccaccctggatgacaatggaaccatgctgttttttaaaggggagtttgtgtggaagagtcacaaatgggaccgggagttaatctcagagagatggaagaatttccccagccctgtggatgctgcattccgtcaaggtcacaacagtgtctttctgatcaagggggacaaagtctgggtataccctcctgaaaagaaggagaaaggatacccaaagttgctccaagatgaatttcctggaatcccatccccactggatgcagctgtggaatgtcaccgtggagaatgtcaagctgaaggcgtcctcttcttccaaggtgaccgcgagtggttctgggacttggctacgggaaccatgaaggagcgttcctggccagctgttgggaactgctcctctgccctgagatggctgggccgctactactgcttccagggtaaccaattcctgcgcttcgaccctgtcaggggagaggtgcctcccaggtacccgcgggatgtccgagactacttcatgccctgccctggcagaggccatggacacaggaatgggactggccatgggaacagtacccaccatggccctgagtatatgcgctgtagcccacatctagtcttgtctgcactgacgtctgacaaccatggtgccacctatgccttcagtgggacccactactggcgtctggacaccagccgggatggctggcatagctggcccattgctcatcagtggccccagggtccttcagcagtggatgctgccttttcctgggaagaaaaactctatctggtccagggcacccaggtatatgtcttcctgacaaagggaggctataccctagtaagcggttatccgaagcggctggagaaggaagtcgggacccctcatgggattatcctggactctgtggatgcggcctttatctgccctgggtcttctcggctccatatcatggcaggacggcggctgtggtggctggacctgaagtcaggagcccaagccacgtggacagagcttccttggccccatgagaaggtagacggagccttgtgtatggaaaagtcccttggccctaactcatgttccgccaatggtcccggcttgtacctcatccatggtcccaatttgtactgctacagtgatgtggagaaactgaatgcagccaaggcccttccgcaaccccagaatgtgaccagtctcctgggctgcactcactga

SEQ ID NO: 16 - DNA, 3260 bp, Homo sapiens
  CDS (37)...(2829)
gagttcagaagcctcctggcagacactggagccacgatgaagcccccaaggcctgtccgtacctgcagcaaagttctcgtcctgctttcactgctggccatccaccagactactactgccgaaaagaatggcatcgacatctacagcctcaccgtggactccagggtctcatcccgatttgcccacacggtcgtcaccagccgagtggtcaatagggccaatactgtgcaggaggccaccttccagatggagctgcccaagaaagccttcatcaccaacttctccatgatcatcgatggcatgacctacccagggatcatcaaggagaaggctgaagcccaggcacagtacagcgcagcagtggccaagggaaagagcgctggcctcgtcaaggccaccgggagaaacatggagcagttccaggtgtcggtcagtgtggctcccaatgccaagatcacctttgagctggtctatgaggagctgctcaagcggcgtttgggggtgtacgagctgctgctgaaagtgcggccccagcagctggtcaagcacctgcagatggacattcacatcttcgagccccagggcatcagctttctggagacagagagcaccttcatgaccaaccagctggtagacgccctcaccacctggcagaataagaccaaggctcacatccggttcaagccaacactttcccagcagcaaaagtccccagagcagcaagaaacagtcctggacggcaacctcattatccgctatgatgtggaccgggccatctccgggggctccattcagatcgagaacggctactttgtacactactttgcccccgagggcctaaccacaatgcccaagaatgtggtctttgtcattgacaagagcggctccatgagtggcaggaaaatccagcagacccgggaagccctaatcaagatcctggatgacctcagccccagagaccagttcaacctcatcgtcttcagtacagaagcaactcagtggaggccatcactggtgccagcctcagccgagaacgtgaacaaggccaggagctttgctgcgggcatccaggccctgggagggaccaacatcaatgatgcaatgctgatggctgtgcagttgctggacagcagcaaccaggaggagcggctgcccgaagggagtgtctcactcatcatcctgctcaccgatggcgaccccactgtgggggagactaaccccaggagcatccagaataacgtgcgggaagctgtaagtggccggtacagcctcttctgcctgggcttcggtttcgacgtcagctatgccttcctggagaagctggcactggacaatggcggcctggcccggcgcatccatgaggactcagactctgccctgcagctccaggacttctaccaggaagtggccaacccactgctgacagcagtgaccttcgagtacccaagcaatgccgtggaggaggtcactcagaacaacttccggctcctcttcaagggctcagagatggtggtggctgggaagctccaggaccgggggcctgatgtgctcacagccacagtcagtgggaagctgcctacacagaacatcactttccaaacggagtccagtgtggcagagcaggaggcggagttccagagccccaagtatatcttccacaacttcatggagaggctctgggcatacctgactatccagcagctgctggagcaaactgtctccgcatccgatgctgatcagcaggccctccggaaccaagcgctgaatttatcacttgcctacagctttgtcacgcctctcacatctatggtagtcaccaaacccgatgaccaagagcagtctcaagttgctgagaagcccatggaaggcgaaagtagaaacaggaatgtccactcaggttccactttcttcaaatattatctccagggagcaaaaataccaaaaccagaggcttccttttctccaagaagaggatggaatagacaagctggagctgctggctcccggatgaatttcagacctggggttctcagctccaggcaacttggactcccaggacctcctgatgttcctgaccatgctgcttaccaccccttccgccgtctggccatcttgcctgcttcagcaccaccagccacctcaaatcctgatccagctgtgtctcgtgtcatgaatatgaaaatcgaagaaacaaccatgacaacccaaaccccagcccccatacaggctccctctgccatcctgccactgcctgggcagagtgtggagcggctctgtgtggaccccagacaccgccaggggccagtgaacctgctctcagaccctgagcaaggggttgaggtgactggccagtatgagagggagaaggctgggttctcatggatcgaagtgaccttcaagaaccccctggtatgggttcacgcatcccctgaacacgtggtggtgactcggaaccgaagaagctctgcgtacaagtggaaggagacgctattctcagtgatgcccggcctgaagatgaccatggacaagacgggtctcctgctgctcagtgacccagacaaagtgaccatcggcctgttgttctgggatggccgtggggaggggctccggctccttctgcgtgacactgaccgcttctccagccacgttggagggacccttggccagttttaccaggaggtgctctggggatctccagcagcatcagatgacggcagacgcacgctgagggttcagggcaatgaccactctgccaccagagagcgcaggctggattaccaggaggggcccccgggagtggagatttcctgctggtctgtggagctgtagttctgatggaaggagctgtgcccaccctgtacacttggcttccccctgcaactgcagggccgcttctggggcctggaccaccatggggaggaagagtcccactcattacaaataaagaaaggtggtgtgagcctgggaagtgggtgtctccagttccatgtggccaaatcctagggcctcaacctcgcatcctgaaccttagcatcgtggaacacagaagcttccactgtcagctctcaagagcccatggccaggaaggcccatgctgagctttcagtccagccccttcattttacaaacaaggaaactgagctcgaaccacccatttgagatgtcactgtggcccccagctagaggcccagggctgggagcattctccaggagcagaggttcagtctgcttcatggtctcttggaccagttttgactaca

SEQ ID NO: 17 - DNA, 1652 bp, Homo sapiens
  CDS (73)...(570)
aaaaggggcgggaggccaggctcgtgccgttttgcagacgccaccgccgaggaaaaccgtgtactattagccatggtcaaccccaccgtgttcttcgacattgccgtcgacggcgagcccttgggccgcgtctcctttgagctgtttgcagacaaggtcccaaagacagcagaaaattttcgtgctctgagcactggagagaaaggatttggttataagggttcctgctttcacagaattattccagggtttatgtgtcagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggttggatggcaagcatgtggtgtttggcaaagtgaaagaaggcatgaatattgtggaggccatggagcgctttgggtccaggaatggcaagaccagcaagaagatcaccattgctgactgtggacaactcgaataagtttgacttgtgttttatcttaaccaccagatcattccttctgtagctcaggagagcacccctccaccccatttgctcgcagtatcctagaatctttgtgctctcgctgcagttccctttgggttccatgttttccttgttccctcccatgcctagctggattgcagagttaagtttatgattatgaaataaaaactaaataacaattgtcctcgtttgagttaagagtgttgatgtaggctttattttaagcagtaatgggttacttctgaaacatcacttgtttgcttaattctacacagtacttagattttttttactttccagtcccaggaagtgtcaatgtttgttgagtggaatattgaaaatgtaggcagcaactgggcatggtggctcactgtctgtaatgtattacctgaggcagaagaccacctgagggtaggagtcaagatcagcctgggcaacatagtgagacgctgtctctacaaaaaataattagcctggcctggtggtgcatgcctagtcctagctgatctggaggctgacgtgggaggattgcttgagcctagagtgagctattatcatgccactgtacagcctgggtgttcacagatcttgtgtctcaaaggtaggcagaggcaggaaaagcaaggagccagaattaagaggttgggtcagtctgcagtgagttcatgcatttagaggtgttcttcaagatgactaatgtcaaaaattgagacatctgttgcggttttttttttttttttttcccctggaatgcagtggcgtgatctcagctcactgcagcctccgcctcctgggttcaagtgattctagtgcctcagcctcctgagtagctgggataacgggcgtgtgccaccatgcccagctaatttttgtatttttagtatagatggggtttcatcattttgaccaggctggtctcaaactcttgacctcagctgatgcgcctgccttggcctcccaaactgctgagattacagatgtgagccaccgcaccctacctcattttctgtaacaaagctaagcttgaacactgttgatgttcttgagggaagcat

SEQ ID NO: 18 - DNA, 1856 bp, Homo sapiens
  CDS (299)...(979)
agccaaaagaggaagggaccggcctcccacgtccacagggacctgacttccacctctctgcccagatttgcttatgtcactgtcgccccgggacggggaggtggggagctgagggcaagtcgcgcccgcccctgaaatcccagccgcctagcgattggctgcaagggtctcggcttggccgcggattaatcacacccgagggcttgaaaggtggctgggagcgccggacacctcagacggacggtggccagggatcaggcagcggctcaggcgaccctgagtgtgcccccaccccgccatggcccggctgctgcaggcgtcctgcctgctttccctgctcctggccggcttcgtctcgcagagccggggacaagagaagtcgaagatggactgccatggtggcataagtggcaccatttacgagtacggagccctcaccattgatggggaggagtacatccccttcaagcagtatgctggcaaatacgtcctctttgtcaacgtggccagctactgaggcctgacgggccagtacattgaactgaatgcactacaggaagagcttgcaccattcggtctggtcattctgggctttccctgcaaccaatttggaaaacaggaaccaggagagaactcagagatccttcctaccctcaagtatgtccgaccaggtggaggctttgtccctaatttccagctctttgagaaaggggatgtcaatggagagaaagagcagaaattctacactttcctaaagaactcctgtcctcccacctcggagctcctgggtacatctgaccgcctcttctgggaacccatgaaggttcacgacatccgctggaactttgagaagttcctggtggggccagatggtatacccatcatgcgctggcaccaccggaccacggtcagcaacgtcaagatggacatcctgtcctacatgaggcggcaggcagccctgggggtcaagaggaagtaactgaaggccgtctcatcccatgtccaccatgtaggggagggactttgttcaggaagaaatccgtgtctccaaccacactatctacccatcacagacccctttcctatcactcaaggccccagcctggcacaaatggatgcatacagttctgtgtactgccaggcatgtgggtgtgggtgcaatgtgggtgtttacacacatgcctacaggtatgcgtgattgtgtgtgtgtgcatgggtgtacagccacgtgtctacctatgtgtctttctgggaatgtgtaccatctgtgtgcctgcagctgtgtagtgctggacagtgacaaccctttctctccagttctccactccaatgataatagttcacttatacctaaacccaaaggaaaaaccagctctaggtccaattgttctgctctaactgatacctcaaccttggggccagcatctcccactgcctccaaatattagtaactatgactgacgtccccagaagtttctgggtctaccacactccccaaccccccactcctacttcctgaagggccctcccaaggctacatccccaccccacagttctccctgagagagatcaacctccctgagatcaaccaaggcagatgtgacagcaagggccacggaccccatggcaggggtggcgtcttcatgagggaggggcccaaagcccttgtgggcggacctcccctgagcctgtctgaggggccagcccttagtgcattcaggctaaggcccctgggcagggatgccacccctgctccttcggaggacgtgccctcacccctcactggtccactggcttgagactcaccccgtctgcccagtaaaagcctttctgcagcaaaaaaaaaaaaaaaaaaa

SEQ ID NO: 19 - DNA, 715 bp, Homo sapiens
  CDS (81)...(467)
tgcagacttgtaggcagcaactcaccctcactcagaggtcttctggttctggaaacaactctagctcagccttctccaccatgagcctcagacttgataccaccccttcctgtaacagtgcgagaccacttcatgccttgcaggtgctgctgcttctgtcattgctgctgactgctctggcttcctccaccaaaggacaaactaagagaaacttggcgaaaggcaaagaggaaagtctagacagtgacttgtatgctgaactccgctgcatgtgtataaagacaacctctggaattcatcccaaaaacatccaaagtttggaagtgatcgggaaaggaacccattgcaaccaagtcgaagtgatagccacactgaaggatgggaggaaaatctgcctggacccagatgctcccagaatcaagaaaattgtacagaaaaaattggcaggtgatgaatctgctgattaatttgttctgtttctgccaaacttctttaactcccaggaagggtagaattttgaaaccttgattttctagagttctcatttattcaggatacctattcttactgtattaaaatttggatatgtgtttcattctgtctcaaaaatcacattttattctgagaaggttggttaaaagatggcagaaagaagatgaaaataaataagcctggtttcaaccctctaattcttgcctaaaaaaaaaaaaaaaaa

SEQ ID NO: 20 - DNA, 2318 bp, Homo sapiens
  CDS (51)...(2147)
gcacagaagcgagtccgactgtgctcgctgctcagcgccgcacccggaagatgaggctcgccgtgggagccctgctggtctgcgccgtcctggggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaaaatctcagaggtagggctgccaccaaggtgaagatgggaacgcagatgatccatgagtttgccctggtttcactggcccaagtggtttgtgctaaccacgtctgtcttcacagctctgtgttgccatgtgtgctgaacaaaaaataaaaattattattgattttatatttc

SEQ ID NO: 21 - DNA, 722 bp, Homo sapiens
  CDS (225)...(593)
aaggctcagtataaatagcagccaccgctccctggcaggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaaaaaaaaaaaaaaaaaaa

SEQ ID NO: 22 - DNA, 614 bp, Homo sapiens
  CDS (76)...(468)
tatagctccacggccagaagataccagcagctctgcctttactgaaatttcagctggagaaaggtccacagcacaatgaggcttttcacaggcattgttttctgctccttggtcatgggagtcaccagtgaaagctggcgttcgtttttcaaggaggctctccaaggggttggggacatgggcagagcctattgggacataatgatatccaatcaccaaaattcaaacagatatctctatgctcggggaaactatgatgctgcccaaagaggacctgggggtgtctgggctgctaaactcatcagccgttccagggtctatcttcagggattaatagactactatttatttggaaacagcagcactgtattggaggactcgaagtccaacgagaaagctgaggaatggggccggagtggcaaagaccccgaccgcttcagacctgacggcctgcctaagaaatactgagcttcctgctcctctgctctcagggaaactgggctgtgagccacacacttctccccccagacagggacacagggtcactgagctttgtgtccccaggaactggtatagggcacctagaggtgttcaataaatgtttgtcaaattga

SEQ ID NO: 23 - DNA, 874 bp, Homo sapiens
  CDS (94)...(702)
gggcgggaagacgtgcagcctgggccgtggctgctcactgcgttcggacccagacccgctgcaggcagcagcagcccccgcccgcgcacgagcatggagctctggggggcctacctcctcctctgcctcttctccctcctgacccaggtcaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctgcagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggcgcccgcatcgcctacaagaactgggagactgagatcaccgcgcaacccgatggcggcaagaccgagaactgcgcggtcctgtcaggcgcggccaacggcaagtggttcgacaagcgctgccgcgatcagctgccctacatctgccagttcgggatcgtgtagccggcggggcgggggccgtggggggcctggaggagggcaggagccgcgggaggccgggaggagggtggggaccttgcagcccccatcctctccgtgcgcttggagcctctttttgcaaataaagttggtgcacgttcgcggagaggaaaaaaaaaaaaaaaaaaaaaaaaaa

SEQ ID NO: 24 - DNA, 615 bp, Homo sapiens
  CDS (27)...(470)
acagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcactgttttcacctcatatgctatgttagaagtccaggcagagacaataaaacattcctgtgaaaggc

SEQ ID NO: 25 - DNA, 2022 bp, Homo sapiens
  CDS (494)...(1930)
caatcatggatcaatagctatgtttggagaaggaatttgtggctgctccagctactgggcattttgtctggtccagttcatgtaatctcccaacaccccatgaagcaaggctttgttaatcctattttactgaaaatgaactaagactcagagagataaagctgttgcccaatgagccttctttctgccctccagatccacggtgctaattccccttccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccatggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccatgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctgtaggagtcagagcccacatggccgggccctctgtagctccctcctcccatctccttcccccagcccaataaaggtcccttagccccgagtttaaa

SEQ ID NO: 26 - DNA, 1166 bp, Homo sapiens
  CDS (7)...(894)
gcaagaatggtgcctgtcctgctgtctctgctgctgcttctgggtcctgctgtcccccaggagaaccaagatggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacattgtggagtattacaacgacagtaacgggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagc

SEQ ID NO: 27 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 28 - protein, 352 aa, Homo sapiens
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 29 - protein, 398 aa, Homo sapiens
MEGAALLRVSVLCIWMSALFLGVRVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 30 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 31 - protein, 261 aa, Homo sapiens
MASPDWGYDDKNGPEQWSKLYPIANGNNQSPVDIKTSETKHDTSLKPISVSYNPATAKEIINVGHSFHVNFEDNDNRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 32 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 33 - protein, 166 aa, Homo sapiens
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 34 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 35 - protein, 270 aa, Homo sapiens
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 36 - protein, 313 aa, Homo sapiens
MELDRAVGVLGAATLLLSFLGMAWALQAADTCPEVKMVGLEGSDKLTILRGCPGLPGAPGDKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 37 - protein, 299 aa, Homo sapiens [sequence omitted]

SEQ ID NO: 38 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 39 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 40 - protein, 348 aa, Homo sapiens
MSDLGAVISLLLWGRQLFALYSGNDVTDISDDRFPKPPEIANGYVEHLFRYQCKNYYRLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQDWVQKTIAEN

SEQ ID NO: 41 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 42 - protein, 930 aa, Homo sapiens [sequence omitted]

SEQ ID NO: 43 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 44 - protein, 226 aa, Homo sapiens
MARLLQASCLLSLLLAGFVSQSRGQEKSKMDCHGGISGTIYEYGALTIDGEEYIPFKQYAGKYVLFVNVASYCGLTGQYIELNALQEELAPFGLVILGFPCNQFGKQEPGENSEILPTLKYVRPGGGFVPNFQLFEKGDVNGEKEQKFYTFLKNSCPPTSELLGTSDRLFWEPMKVHDIRWNFEKFLVGPDGIPIMRWHHRTTVSNVKMDILSYMRRQAALGVKRK

SEQ ID NO: 45 - protein, 128 aa, Homo sapiens
MSLRLDTTPSCNSARPLHALQVLLLLSLLLTALASSTKGQTKRNLAKGKEESLDSDLYAELRCMCIKTTSGIHPKNIQSLEVIGKGTHCNQVEVIATLKDGRKICLDPDAPRIKKIVQKKLAGDESAD

SEQ ID NO: 46 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 47 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 48 - protein, 130 aa, Homo sapiens
MRLFTGIVFCSLVMGVTSESWRSFFKEALQGVGDMGRAYWDIMISNHQNSNRYLYARGNYDAAQRGPGGVWAAKLISRSRVYLQGLIDYYLFGNSSTVLEDSKSNEKAEEWGRSGKDPDRFRPDGLPKKY

SEQ ID NO: 49 - protein, 202 aa, Homo sapiens
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 50 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 51 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 52 - protein, 295 aa, Homo sapiens
MVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS